(19) United States
(12) Patent Application Publication
(10) Pub. No.: US 2026/0090970 A1
(43) Pub. Date: Apr. 2, 2026

(54) POLYTERPENE FILM FORMER FOR COSMETIC APPLICATIONS

(71) Applicant: Momentive Performance Materials Inc., Niskayuna, NY (US)

(72) Inventors: Roy U. ROJAS WAHL, Saddle River, NJ (US); Benjamin FALK, Yorktown Heights, NY (US); Koushik MUKHERJEE, Bangalore (IN); Rajendar NUTENKI, Telangana (IN); Amar PAWAR, Briarcliff Manor, NY (US); Sibin WANG, Woodside, NY (US)

(21) Appl. No.: 19/342,096

(22) Filed: Sep. 26, 2025

(30) Foreign Application Priority Data
Sep. 28, 2024 (IN) 202411073498

(51) Int. Cl. A61K 8/81; A61Q 19/00; C08F 110/14; C08F 136/04

(52) U.S. Cl. CPC A61K 8/8111; A61Q 19/00; C08F 110/14; C08F 136/045; A61K 2800/10

(57) ABSTRACT

The present disclosure provides a personal care composition comprising at least one terpene polymer with a weight average molecular weight greater than 3,000 g/mol that when formulated into the composition for personal care, delivers color transfer resistance, even when exposed to water, and/or food oil. The personal care composition comprising at least one terpene polymer can be combined with components typically used in personal care to provide personal care formulations.

### FIELD

The present disclosure provides a personal care composition comprising at least one terpene polymer with a weight average molecular weight greater than 3,000 g/mol that when formulated into the composition for personal care, delivers color transfer resistance, even when exposed to oil, water, and/or food. The personal care composition comprising at least one terpene polymer can be combined with components typically used in personal care to provide personal care formulations.

### BACKGROUND

There exists a need in the market for non-silicone, non-acrylate, 100% natural film formers for use in cosmetic applications, especially in lip-color and lipstick, providing resistance to (a) general color transfer, (b) color transfer when exposed to food oils, and/or (c) color transfer when exposed to beverages containing water.

Natural resins of plant or animal origin constitute an advantageous product class by virtue of their special properties linked to nature. However, such resins of natural origin are rarely used in cosmetic compositions because they are generally insoluble in water, a ubiquitous solvent used in the cosmetic industry. Attempts have been made to adapt these natural resins to a form that is suitable for use with water.

Recently a variety of terpenes and their derivatives have been investigated as natural sources of monomers, to produce various classes of polymers, such as poly(myrcene), poly(limonene), and poly(β-pinene). These terpene polymers are used as tackifier resins in adhesives and are synthesized as pallets or as irregularly shaped powders.

U.S. Pat. No. 3,314,981 discloses the polymerization of turpentine or fractions thereof to provide liquid terpene polymers for use in cosmetics as softening agents. These liquid terpene polymers have a molecular weight below about 500 g/mol. U.S. Pat. No. 8,252,270 discloses terpene resins having a molecular weight from about 300 g/mol to about 2,000 g/mol used to prepare cosmetic compositions. These disclosures did not investigate the transfer resistance of these terpene polymers, let alone investigate the effect of a terpene polymer having a molecular weight greater than 3,000 g/mol on transfer resistance after exposure to food oils.

There is a need to provide polymers derived from plant-based monomers that can be formulated in a hydrophobic environment, such as an environment that typically uses a volatile hydrocarbon (e.g., isododecane) as a solvent. There is a need to provide polymers derived from plant-based monomers that provide improved transfer resistance, water resistance, and food oil resistance.

### SUMMARY

The present disclosure provides a personal care composition comprising at least one terpene polymer with a weight average molecular weight greater than 3,000 g/mol.

In some aspects, the at least one terpene polymer is obtained by polymerizing a terpene monomer selected from the group consisting of a monoterpene, a sesquiterpene, a diterpene, a sesterterpene, a triterpene, a sesquaterpene, a tetraterpene, and combinations thereof.

In some aspects, the terpene monomer is a monoterpene selected from the group consisting of myrcene, ocimene, alloocimene, L-limonene, D-limonene, α-pinene, and β-pinene.

In some aspects, the at least one terpene polymer is a homopolymer obtained by polymerizing a terpene monomer selected from the group consisting of α-pinene, β-pinene, L-limonene, and D-limonene.

In some aspects, the at least one terpene polymer is a copolymer obtained by polymerizing at least one terpene monomer selected from the group consisting of α-pinene, β-pinene, L-limonene, D-limonene, and combinations thereof.

In some aspects, at least one terpene monomer comprises at least 50% of the total weight of monomers used to obtain the copolymer.

In some aspects, at least one terpene monomer comprises at least 95% of the total weight of monomers used to obtain the copolymer.

In some aspects, the at least one terpene polymer is a homopolymer of β-pinene.

In some aspects, the terpene polymer is hydrogenated to a degree from about 5% to 100%.

In some aspects, the at least one terpene polymer is a copolymer obtained by polymerizing at least one terpene monomer and at least one monomer that is not a terpene.

In some aspects, the at least one terpene polymer has a weight average molecular weight from about 3,500 g/mol to about 100,000 g/mol.

In some aspects, the at least one terpene polymer has a weight average molecular weight from about 5,000 g/mol to about 50,000 g/mol.

In some aspects, the at least one terpene polymer has a weight average molecular weight from about 10,000 g/mol to about 20,000 g/mol.

In some aspects, the at least one terpene polymer has a glass transition temperature from about 50° C. to about 300° C.

In some aspects, the at least one terpene polymer has a glass transition temperature from about 50° C. to about 150° C.

In some aspects, the personal care composition further comprises at least one volatile oil.

In some aspects, the volatile oil is selected from the group consisting of silicone oil, hydrocarbon oil, and mixtures thereof.

In some aspects, the volatile oil is selected from the group consisting of cyclopentasiloxane, methyl trimethicone, dimethicone, ethyl trisiloxane, isododecane, isohexadecane, n-dodecane, n-decane, n-undecane, n-tridecane, isoparaffin, linear or branched alkanes from carbon chain length C₉ to C₁₆, and mixtures thereof.

In some aspects, the personal care composition further comprises a colorant.

In some aspects, the personal care composition further comprises a colorant, such as a pigment or a dye.

In some aspects, the personal care composition comprises a volatile oil and a pigment.

In some aspects, the personal care composition further comprises a non-volatile oil.

In some aspects, the non-volatile oil is selected from the group consisting of an aliphatic hydrocarbon, a polyisobutylene, a polyethylene, a silicone oil, a silicone gum, a butter, a lipid, a fatty acid, an alcohol, a ceramide, an ether, a linear or branched hydrocarbon, a linear or branched ester, a vegetable oil, and combinations and/or oligomers thereof.

In some aspects, the personal care composition is anhydrous.

In some aspects, the personal care composition is an oil-in-water emulsion or a water-in-oil emulsion.

In some aspects, the personal care composition further comprises at least one elastomer.

In some aspects, the elastomer is a crosslinked elastomer.

In some aspects, the crosslinked elastomer is capable of swelling and absorbing at least 20% by weight of a solvent fluid.

In some aspects, the crosslinked elastomer has a particle size diameter from about 10 microns to about 200 microns.

In some aspects, the personal care composition further comprises a wax.

In some aspects, the personal care composition further comprises a rheology modifier.

In some aspects, the personal care composition further comprises at least one volatile oil, at least one elastomer, at least one rheology modifier, and at least one pigment or dye.

In some aspects, the weight of the at least one terpene polymer in the composition is from about 0.1% to about 30% of the total weight of the composition.

In some aspects, the personal care composition is capable of forming a film on a keratinous substrate.

In some aspects, the film is capable of remaining on the keratinous substrate for at least one hour without flaking or transferring to an object that comes in contact with the film.

In some aspects, the film has a resistance to food oil of greater than or equal to about 50% as measured by color transfer.

In some aspects, the personal care formulation is a deodorant, an antiperspirant, a skin cream, a facial cream, a hair shampoo, a hair conditioner, a mousse, a hair styling gel, a hair spray, a protective cream, a lipstick, a lip color, a lip stain, a lip gloss, a facial foundation, a concealer, blushes, makeup, a mascara, a BB cream, a CC cream, a skin care lotion, a moisturizer, a facial treatment, a personal cleanser, a facial cleanser, a bath oil, a perfume, a shaving cream, a pre-shave lotion, an after-shave lotion, a cologne, a sachet, or a sunscreen.

In some aspects, the personal care composition is used in the manufacture of a personal care formulation.

The present disclosure also provides a terpene polymer obtained by polymerizing a terpene monomer selected from the group consisting of α-pinene, β-pinene, L-limonene, D-limonene, and combinations thereof, wherein the terpene polymer has a molecular weight of greater than 3,000 g/mol.

### BRIEF DESCRIPTION OF THE DRAWINGS

FIG. 1 shows the transfer resistance (“TR”), water resistance (“WR”), and food oil resistance (“OR”) results of Formulations 1-3 in TABLE 1. Formulation 1 is a benchmark formulation that does not comprise a terpene polymer but does comprise a dimethicone gum and a silicone resin, Formulation 2 comprises poly(β-pinene) having a weight average molecular weight of about 8,291 g/mol that also comprised a dimethicone gum and a silicone resin, and Formulation 3 is a blank formulation that does not comprise a terpene polymer, does not comprise a dimethicone gum, and does not comprise a silicone resin.

FIG. 2 shows the transfer resistance (“TR”), water resistance (“WR”), and food oil resistance (“OR”) results of Formulations 5 and 6 in TABLE 1. Formulation 4 comprises poly(β-pinene) having a weight average molecular weight of about 5,739 g/mol and that also comprises a dimethicone gum and a silicone resin and Formulation 5 comprises poly(β-pinene) having a weight average molecular weight of about 2,641 g/mol that also comprises a dimethicone gum and a silicone resin.

FIG. 3 shows the transfer resistance (“TR”), water resistance (“WR”), and food oil resistance (“OR”) results of Formulations 6 and 7 in TABLE 1. Formulation 6 comprises poly(limonene) that also comprises a dimethicone gum and a silicone resin and Formulation 7 is a blank formulation that does not comprise a terpene polymer, does not comprise a dimethicone gum, and does not comprise a silicone resin.

FIG. 4 shows the transfer resistance (“TR”), water resistance (“WR”), and food oil resistance (“OR”) results of Formulations 8 and 9 in TABLE 1. Formulation 8 comprises a commercially available copolymer of α-pinene and β-pinene with a softening point of 115° C. and also comprises a dimethicone gum and a silicone resin and Formulation 9 comprises a commercially available copolymer of α-pinene and β-pinene with a softening point of 125° C. and also comprises a dimethicone gum and a silicone resin.

FIG. 5 shows the transfer resistance (“TR”), water resistance (“WR”), and food oil resistance (“OR”) results of Formulation 10 in TABLE 1. Formulation 10 comprises a commercially available hydrogenated poly(limonene) and also comprises a dimethicone gum and a silicone resin.

FIG. 6 shows Dynamic Mechanical Analysis (DMA) curves measuring the glass transition temperature (Tg) for formulations comprising different ratios of plasticizer (polyisobutylene (PIB)) to film former (poly ß-pinene (PBP)) which provide the loss factor as a function of temperature.

### DETAILED DESCRIPTION

### I. Definitions

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the region of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

All ranges disclosed herein are inclusive of the endpoints, and the endpoints are independently combinable with each other. Each range disclosed herein constitutes a disclosure of any point or sub-range lying within the disclosed range. For example, the range from X to Y, is inclusive of X and Y. And, the range between X and Y, is inclusive of X and Y.

As used herein, the following definitions shall apply unless otherwise indicated. For purposes of the present disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry,” Thomas Sorrell, University Science Books, Sausalito: 1999, and “March's Advanced Organic Chemistry,” 6^{th} Ed., Smith, M. B. and March, J., eds. John Wiley & Sons, New York: 2007, the entire contents of which are hereby incorporated by reference in their entireties.

The phrase “personal care composition” as used herein, refers to a chemical mixture comprising a composition comprising polymer particles and additional components used for the purpose of cleansing, conditioning, grooming, beautifying, or otherwise enhancing the appearance of the human body.

The phrase “personal care formulation” as used herein, refers to a product marketed for use in its final form comprising a composition of polymer particles and/or a personal care composition, where the product has been further formulated for application to a human body. The personal care formulation can be in the form of a liquid, a gel, a foaming gel, a lotion, a cream, a paste, a powder, a bar, a stick, an emulsion, a dispersion, a suspension, a spray, or other commonly used forms in the industry.

Terpenes are classified using the isoprene rule (C5 rule), which is based on the number and organization of carbon atoms in the molecule, thus, grouping these molecules according to the number of isoprene units linked in a head-to-tail manner and constituting hemiterpenes (1 isoprene unit), monoterpenes (2 isoprene units; C₁₀H₁₆), sesquiterpenes (3 isoprene units; C₁₅H₂₄), and diterpenes (4 isoprene units; C₂₀H₃₂), or in general, compounds with formula (C₅H₈)ₙ, wherein n≥2. Although the head-to-tail configuration is the most common, non-head-to-tail condensation of isoprene units also exists. The term “monomer” as used herein is an organic that can be us used as a raw material in a polymerization reaction, and which becomes part of a polymer backbone during the polymerization reaction.

The term “polymer” as used herein, refers to a macromolecular structure comprising repeating molecular arrangements of monomers. The term polymer is intended to encompass polymers that contain more than three repeating units.

The term “homopolymer” as used herein, refers to a polymer made from repeating units of a single monomer.

The term “copolymer” as used herein, refers to a polymer made from repeating units of at least two different monomers.

The term “hydrogenated” as used herein, refers to a chemical process that adds hydrogen atoms to unsaturated (double) bonds to form saturated (single) bonds. In some aspects, the extent of hydrogenation is from about 0.01% to 100%.

The weight average molecular weight (M_{W}) is calculated from the weight fraction distribution of different sized molecules. Since larger molecules m a sample weigh more than smaller molecules, the M_{W} is skewed to higher values, and is always greater than the number average molecular weight (Mₙ).

$${M}_{w} = \sum {w}_{i} {M}_{i} \\ {M}_{n} = \sum {n}_{i} {M}_{i}$$

wherein:
wᵢ=weight fraction of chains with molecular weight Mᵢ(wᵢ=(nᵢMᵢ)/Mₙ)
nᵢ=mole fraction of chains with molecular weight Mᵢ

The term “oligomer” as used herein, refers to a compound made of repeating molecular units of monomers. Unlike polymers, oligomers have a much lower molecular weight since they are made up of only a few repeating units. Oligomers are classified by the number of monomers for example, a tetramer is comprised of four monomers. Similar to polymers, oligomers can be made from identical or non-identical monomers.

The phrase “BB cream” as used herein, refers to a “blemish balm” or a “beauty balm” and is used to moisturize, protect, and/or correct the skin.

The phrase “CC cream” as used herein, refers to a “color corrector” or a “complexion corrector” and is used to neutralize the look of skin imperfections by utilizing colors opposite of each other on the color wheel. For example, to correct purple under eye circles and veins, a CC cream with a peach/orange color can be used or to correct redness associated with rosacea, sunburn, acne, or broken capillaries, a green CC cream can be used.

The phrase “rheology modifier” as used herein, refers to a component used to not only thicken and modify the viscosity of a personal care composition but also to structure the composition so that when a force is applied it becomes flowable and easily poured. Examples of rheology modifiers include natural gums, cellulose, clays, polyethylene glycols, synthetic polymers, and organoclays.

The term “colorant (A)” preferably refers to the group including:
1) untreated pigments;
2) treated pigments;
3) dyes;
4) pearls; and
combinations thereof.

The term “pigment” refers to white or colored, mineral or organic particles, insoluble in aqueous medium, intended to color and/or opacify the composition. Since they are insoluble, they remain suspended and contribute color through dispersion and suspension rather than solvation. Pigments are often used in cosmetic compositions such as foundations, concealers, lipsticks, lip stains, lip liners, blushes, eyeshadows, mascara, eyeliners, brow gels, sunscreens, and more. Pigments often contribute to cosmetic compositions' long-lasting properties and minimal migration.

In some aspects, cosmetic compositions contain one or more pigments, which are typically present in a different phase from the gel phase. The colorant such as pigment used herein can be inorganic and/or organic. Cosmetic compositions according to the invention preferably comprise greater than or equal to 0.1% colorants such as pigments by weight of the cosmetic composition to provide a coloring or pigmenting effect. Typically, the colorants such as pigments may be present from about 0.1% to 25%, preferably from about 0.5 to 15%, and most preferably from about 1 to 10% by weight.

Pigments can be selected from the group consisting of titanium oxides such as rutile titanium dioxide, anatase titanium dioxide, zinc oxide, zirconium oxide, iron oxides such as ferric oxide, ferrous oxide, yellow iron oxide, and red iron oxide, bismuth oxychlorides, black iron oxide, acyl glutamate iron oxides, chromium oxide, chromium hydroxide, manganese violet, cerium oxide, ultramarine blue, carmine, and derivatives and mixtures thereof. More preferably, the pigment is titanium oxide, yellow iron oxide, red iron oxide, black iron oxide, and mixtures thereof. The pigments can be surface modified to render them either hydrophobic or hydrophilic to interact synergistically with the particle network.

The term “pearls” refers to pearlescent agents in a cosmetic composition that add optical shimmer and luster or for tactile silkiness to the touch. They are often used in eyeshadows, nail polishes, and highlighters to add sparkle and shine.

Pearls can be selected from the group consisting of synthetic fluorphlogopite, including synthetic fluorphlogopite (and) titanium dioxide (and) iron oxides (and) tin oxide, iron oxides (and) synthetic fluorphlogopite, synthetic fluorphlogopite (and) titanium dioxide (and) tin oxide, synthetic fluorphlogopite (and) iron oxides (and) tin oxide, synthetic fluorphlogopite (and) titanium dioxide (and) tin oxide, calcium sodium borosilicate (and) titanium dioxide (and) tin oxide, calcium sodium borosilicate (and) iron oxides (and) tin oxide, and calcium sodium borosilicate (and) titanium dioxide (and) tin oxide.

The term “dyes” refers to colorants that are soluble in water or other organic solvents. Dyes can be selected from the group consisting of oil-soluble dyes such as green 6, violet 2, and red 17, and water-soluble dyes such as orange 4, red 28 lake, red 33, yellow 10, blue 1, and yellow 8.

The term “colorant dispersion” such as “pigment dispersion”, in beauty and personal care, refers to the process of distributing pigments, often insoluble, into a liquid-based formulation, in order to make colored cosmetic products. Dispersion is extremely important and requires more than simple mixing, such as wetting, separating, and distributing the pigments.

The term “colorant suspension” such as “pigment suspension”, in beauty and personal care, refers to the stabilization of insoluble pigments in a formulation once they have been dispersed.

### Types of Colorants (A)

The following TABLEs 1 to 4 show four categories of colorants used in beauty and personal care: 1) untreated pigments; 2) treated pigments; 3) dyes; and 4) pearls. These are not fully comprehensive lists of all available pigments/dyes but they include examples of each type of pigment and dye used in the industry.

### 1. Untreated Pigments

TABLE 1
Untreated Pigments Used in Beauty and Personal Care

| | | Colour | | | | | | |
|---|---|---|---|---|---|---|---|---|
| | | Index | Max | Restricted | Regions | | Dye | |
| | INCI | (CI) | % | Use Area | Allowed | Trade Name | % | Supplier |
| Organic | Blue 1 Lake | 42090 | N/A | N/A | Global | SunCROMA ® | 12 | Sun |
| | | | | | | FD&C Blue 1 Al | | Chemical |
| | | | | | | Lake C39-4433 | | |
| | Red 21 | 453380 | N/A | Eye | Global | SunCROMA ® | 98 | Sun |
| | | | | | | D&C Red 21 C14- | | Chemical |
| | | | | | | 032 | | |
| | Red 21 Al | 15850 | N/A | Eye | Global | SunCROMA ® | 18 | Sun |
| | Lake | | | | except | D&C Red 21 Al | | Chemical |
| | | | | | Japan | Lake C14-034 | | |
| | Red 22 Lake | 45380 | N/A | Eye | Global | SunCROMA ® | 17 | Sun |
| | | | | | | D&C Red 22 Al | | Chemical |
| | | | | | | Lake C14-6634 | | |
| | Red 27 Lake | 45410 | N/A | Eye | Global | SunCROMA ® | 20 | Sun |
| | | | | | except | D&C Red 27 Al | | Chemical |
| | | | | | Japan | Lake C14-023 | | |
| | Red 28 Lake | 45410 | N/A | N/A | Global | SunCROMA ® | 22 | Sun |
| | | | | | | D&C Red 28 Al | | Chemical |
| | | | | | | Lake C14-6623 | | |
| | Red 30 Lake | 73360 | N/A | Eye | Global | SunCROMA ® | 37 | Sun |
| | | | | | | D&C Red 30 Al | | Chemical |
| | | | | | | Lake C37-038 | | |
| | Red 33 Lake | 17200 | 3% | Eye | Global | SunCROMA ® | 16 | Sun |
| | | | (US, | | | D&C Red 33 Al | | Chemical |
| | | | lip) | | | Lake C17-6444 | | |
| | Red 34 Lake | 15880 | N/A | N/A | Global | SunCROMA ® | 71 | Sun |
| | | | | | | D&C Red 34 Ca | | Chemical |
| | | | | | | Lake C24-A518 | | |
| | Red 36 | 12085 | 3% | Eye | Global | SunCROMA ® | 95 | Sun |
| | | | (US, | | | D&C Red 36 C23- | | Chemical |
| | | | EU) | | | 009 | | |
| | Red 40 | 16035 | N/A | N/A | Global | SunCROMA ® | 37 | Sun |
| | | | | | except | FD&C Red 40 Al | | Chemical |
| | | | | | Japan | Lake C37-6340 | | |
| | Red 6 Lake | 15850 | N/A | Eye | Global | SunCROMA ® | 63 | Sun |
| | | | | | except | D&C Red 6 Ba | | Chemical |
| | | | | | Japan | Lake C19-008 | | |
| | Red 6 Lake | 15850 | N/A | Eye | Global | SunCROMA ® | 95 | Sun |
| | | | | | | D&C Red 6 Sodium | | Chemical |
| | | | | | | Salt C19-6619 | | |
| | Red 7 Lake | 15850 | N/A | Eye | Global | SunCROMA ® | 60 | Sun |
| | | | | | | D&C Red 7 Ca | | Chemical |
| | | | | | | Lake C19-003 | | |
| | Yellow 10 | 47005 | N/A | Eye | Global | SunCROMA ® | 19 | Sun |
| | | | | | except | D&C Yellow 10 Al | | Chemical |
| | | | | | EU | Lake C71-5171 | | |
| | Yellow 5 | 19140 | N/A | N/A | Global | SunCROMA ®FD& | 36 | Sun |
| | Lake | | | | | C Yellow 5 Al Lake | | Chemical |
| | | | | | | C69-4537 | | |
| | Yellow 6 | 15985 | N/A | Eye | Global | SunCROMA ® | 36 | Sun |
| | Lake | | | | | FD&C Yellow 6 Al | | Chemical |
| | | | | | | Lake C70-5270 | | |
| Inorganic | Black Iron | 77499 | N/A | N/A | Global | SunCROMA ® | | Sun |
| | Oxide | | | | | Black Iron Oxide | | Chemical |
| | | | | | | C33-5198 | | |
| | Red Iron | 77491 | N/A | N/A | Global | SunCROMA ® Red | | Sun |
| | Oxide | | | | | Iron Oxide C33- | | Chemical |
| | | | | | | 8075 | | |
| | Yellow Iron | 77492 | N/A | N/A | Global | SunCROMA ® | | Sun |
| | Oxide | | | | | Yellow Iron Oxide | | Chemical |
| | | | | | | C33-8073 | | |
| | Titanium | 77891 | N/A | N/A | Global | SunCROMA ® | | Sun |
| | Dioxide | | | | | Titanium Dioxide | | Chemical |
| | | | | | | C47-088 | | |
| | Chromium | 77288 | N/A | Lip | Global | SunCROMA ® | | Sun |
| | Oxide Green | | | | | Chrome Oxide | | Chemical |
| | | | | | | Green C61-1245 | | |
| | Manganese | 77742 | N/A | N/A | Global | SunCROMA ® | | Sun |
| | Violet | | | | | Manganese Violet | | Chemical |
| | | | | | | C43-001 | | |
| | Ultramarine | 77007 | N/A | Lip | Global | SunCROMA ® | | Sun |
| | Blue | | | | | Ultramarine Blue | | Chemical |
| | | | | | | C43-1810 | | |
| | Ultramarine | 77007 | N/A | Lip | Global | SunCROMA ® | | Sun |
| | Pink | | | | | Ultramarine Pink | | Chemical |
| | | | | | | C43-4875 | | |
| | Ultramarine | 77007 | N/A | Lip | Global | SunCROMA ® | | Sun |
| | Violet | | | | | Ultramarine Violet | | Chemical |
| | | | | | | C43-4888 | | |

### 2. Treated Pigments

TABLE 2
Treated Pigments Used in Beauty and Personal Care

| | | | | Regions | | |
|---|---|---|---|---|---|---|
| INCI Name | Treated With | Blend With | Natural? | approved | Trade Name | Supplier |
| Iron Oxides (CI 77491) (And) | Stearoyl Glutamic Acid | Polyhydroxystearic acid | Yes | Global except | BWRO-ASGP3 | Kobo |
| Stearoyl Glutamic Acid | | | | Australia | | |
| (And) Polyhydroxystearic | | | | | | |
| Acid | | | | | | |
| Titanium Dioxide (And) | Stearoyl Glutamic Acid | Polyhydroxystearic acid | Yes | Global except | BTD-100-ASGP4 | Kobo |
| Stearoyl Glutamic Acid | | | | Australia | | |
| (And) Polyhydroxystearic | | | | | | |
| Acid | | | | | | |
| Blue 1 Lake (And) Stearoyl | Stearoyl Glutamic Acid | | Yes | Global except | BLUE 1AL-ASG4 | Kobo |
| Glutamic Acid | | | | Australia | | |
| Iron Oxides (CI 77491) (And) | Stearoyl Glutamic Acid | | Yes | Global except | BRO-ASG3 | Kobo |
| Stearoyl Glutamic Acid | | | | Australia | | |
| Iron Oxides (CI 77491) (And) | Hydrogenated Olive Oil | | Yes | Global | BRO-NOE4 | Kobo |
| Hydrogenated Olive Oil | Stearyl Esters | | | | | |
| Stearyl Esters | | | | | | |
| Titanium Dioxide (And) | Hydrogenated Olive Oil | | Yes | Global | BTD-NOE2 | Kobo |
| Hydrogenated Olive Oil | Stearyl Esters | | | | | |
| Stearyl Esters | | | | | | |
| Iron Oxides (CI 77491) (And) | Jojoba Esters | Polyhydroxystearic acid | Yes | Australia | BRO-NJEP3 | Kobo |
| Jojoba Esters (And) | | | | | | |
| Polyhydroxystearic Acid | | | | | | |
| Titanium Dioxide (And) | Jojoba Esters | Polyhydroxystearic acid | Yes | Australia | TTO-NJEP8 | Kobo |
| Alumina (And) Jojoba Esters | | | | | | |
| (And) Polyhydroxystearic | | | | | | |
| Acid | | | | | | |
| Iron Oxides (CI 77499) (And) | Jojoba Esters | | Yes | Global | BBO-NJE2 | Kobo |
| Jojoba Esters | | | | | | |
| Titanium Dioxide (And) | Jojoba Esters | | Yes | Global | BTD-NJE2 | Kobo |
| Jojoba Esters | | | | | | |
| Iron Oxides (CI 77491) (And) | Magnesium Myristate | | Yes | Global | BRO/MM3 | Kobo |
| Magnesium Myristate | | | | | | |
| Chromium Hydroxide Green | Magnesium Myristate | | Yes | Global | BHG/MM3 | Kobo |
| (And) Magnesium Myristate | | | | | | |
| Titanium Dioxide (And) | Magnesium Myristate | | Yes | Global | BTD-V-MM3 | Kobo |
| Magnesium Myristate | | | | | | |
| Iron Oxides (CI 77499) (And) | Silica | | Yes | US & Canada | SIH-2 BLACK | Kobo |
| Silica | | | | | No. 710P | |
| Titanium Dioxide (And) | Silica | Aluminum Hydroxide | Yes | US & Canada | SIH-5 TiO2 | Kobo |
| Silica (And) Aluminum | | | | | R250 | |
| Hydroxide | | | | | | |
| Iron Oxides (CI 77491) (And) | Isopropyl Titanium | Triethoxysilylethyl | No | Global | BGRO-TTB2 | Kobo |
| Isopropyl Titanium | Triisostearate | Polydimethylsiloxyethyl | | | | |
| Triisostearate (And) | | Dimethicone | | | | |
| Triethoxysilylethyl | | | | | | |
| Polydimethylsiloxyethyl | | | | | | |
| Dimethicone | | | | | | |
| Titanium Dioxide (And) | Isopropyl Titanium | Triethoxysilylethyl | No | Global | BTD-TTB2 | Kobo |
| Isopropyl Titanium | Triisostearate | Polydimethylsiloxyethyl | | | | |
| Triisostearate (And) | | Dimethicone | | | | |
| Triethoxysilylethyl | | | | | | |
| Polydimethylsiloxyethyl | | | | | | |
| Dimethicone | | | | | | |
| Iron Oxides (CI 77491) (And) | Triethoxycaprylylsilane | Polyhydroxystearic acid | No | Global | BWRO-11SP | Kobo |
| Triethoxycaprylylsilane | | | | | (C33-8001) | |
| (And) Polyhydroxystearic | | | | | | |
| Acid | | | | | | |
| Titanium Dioxide (And) | Triethoxycaprylylsilane | Polyhydroxystearic acid | No | Global | BTD-11SP | Kobo |
| Triethoxycaprylylsilane | | | | | | |
| (And) Polyhydroxystearic | | | | | | |
| Acid | | | | | | |
| Iron Oxides (CI 77491) (And) | Isopropyl Titanium | | No | Global | BRO-12 | Kobo |
| Isopropyl Titanium | Triisostearate | | | | | |
| Triisostearate | | | | | | |
| Titanium Dioxide (And) | Isopropyl Titanium | | No | Global | BTD-100-12 | Kobo |
| Isopropyl Titanium | Triisostearate | | | | | |
| Triisostearate | | | | | | |
| Red 7 Lake (And) Isopropyl | Isopropyl Titanium | | No | Global | RED 7CA K-12 | Kobo |
| Titanium Triisostearate | Triisostearate | | | | | |
| Iron Oxides (CI 77499) (And) | Isopropyl Titanium | Sodium Lauroyl | No | US & Canada | ASI BLACK BL- | Kobo |
| Isopropyl Titanium | Triisostearate | Aspartate (And) Zinc | | | 100P | |
| Triisostearate (And) Sodium | | Chloride | | | | |
| Lauroyl Aspartate (And) Zinc | | | | | | |
| Chloride | | | | | | |
| Titanium Dioxide (And) | Isopropyl Titanium | Sodium Lauroyl | No | US & Canada | ASI TiO2 CR-50 | Kobo |
| Aluminum Hydroxide (And) | Triisostearate | Aspartate (And) Zinc | | | | |
| Isopropyl Titanium | | Chloride | | | | |
| Triisostearate (And) Sodium | | | | | | |
| Lauroyl Aspartate (And) Zinc | | | | | | |
| Chloride | | | | | | |
| Iron Oxides (CI 77491) (And) | Triethoxycaprylylsilane | | No | Global except | BRO-11S2 | Kobo |
| Triethoxycaprylylsilane | | | | Japan | | |
| Titanium Dioxide (And) | Triethoxycaprylylsilane | | No | Global | BTD-100-11S3 | Kobo |
| Triethoxycaprylylsilane | | | | | | |
| Iron Oxides (CI 77491) (And) | Methicone | | No | Global | BXRO-MS2 | Kobo |
| Methicone | | | | | | |
| Titanium Dioxide (And) | Methicone | | No | Global | BTD-MS2 | Kobo |
| Methicone | | | | | | |
| Iron Oxides (CI 77499) (And) | Dimethicone | | No | Global | BBO-DS3 | Kobo |
| Dimethicone | | | | | | |
| Titanium Dioxide (And) | Dimethicone | | No | Global | BTD-DS4 | Kobo |
| Dimethicone | | | | | | |
| Titanium Dioxide (And) | Perfluorohexylethyl | | No | Global | BTD-FS | Kobo |
| Perfluorohexylethyl | Triethoxysilane | | | | | |
| Triethoxysilane | | | | | | |
| Ultramarines (And) | Perfluorohexylethyl | | No | Global | BEUB-FS | Kobo |
| Perfluorohexylethyl | Triethoxysilane | | | | | |
| Triethoxysilane | | | | | | |
| Titanium Dioxide (and) | Algin | Aluminum Hydroxide | Yes | | MiyoAQUA Whte | Miyoshi |
| Aluminum Hydroxide (and) | | | | | TSR | |
| Algin | | | | | | |
| Iron Oxides (and) Algin | Algin | | Yes | | MiyoAQUA Red | Miyoshi |
| Iron Oxides (and) Isostearic | Isostearic Acid | | Yes | | MiyoNAT ISA | Miyoshi |
| Acid | | | | | Yellow-2 | |
| Titanium Dioxide (and) | Isostearic Acid | | Yes | | MiyoNAT ISA | Miyoshi |
| Isostearic Acid | | | | | White A2 | |
| Titanium Dioxide (and) | Hydrogenated Lecithin | Magnesium Chloride | Yes | | MiyoNAT VLS- | Miyoshi |
| Hydrogenated Lecithin (and) | | | | | White A2 | |
| Magnesium Chloride | | | | | | |
| Iron Oxides (and) | Hydrogenated Lecithin | Magnesium Chloride | Yes | | MiyoNAT VLS- | Miyoshi |
| Hydrogenated Lecithin (and) | | | | | Red-2 | |
| Magnesium Chloride | | | | | | |
| Iron Oxides (CI 77499) (and) | Isopropyl Titanium | Bis-PEG-15 | No | APAC | UNIPURE BLACK | Sensient |
| Isopropyl Titanium | Triisostearate | Dimethicone/IPDI | | | LC990 ADT-3 | |
| Triisostearate (and) Bis-PEG- | | Copolymer (and) PEG-2 | | | | |
| 15 Dimethicone/IPDI | | Soyamine | | | | |
| Copolymer (and) PEG-2 | | | | | | |
| Soyamine | | | | | | |
| Iron Oxides (CI 77491) (and) | Isopropyl Titanium | Bis-PEG-15 | No | APAC | UNIPURE RED | Sensient |
| Isopropyl Titanium | Triisostearate | Dimethicone/IPDI | | | LC389 ADT-3 | |
| Triisostearate (and) Bis-PEG- | | Copolymer (and) PEG-2 | | | | |
| 15 Dimethicone/IPDI | | Soyamine | | | | |
| Copolymer (and) PEG-2 | | | | | | |
| Soyamine | | | | | | |
| Iron Oxides (CI 77492) (and) | Persea Gratissima | Hydrogenated Vegetable | Yes | Global | UNIPURE | Sensient |
| Persea Gratissima (Avocado) | (Avocado) Oil | Oil (and) Tocopherol | | | YELLOW LC182 | |
| Oil (and) Hydrogenated | | | | | BA | |
| Vegetable Oil (and) | | | | | | |
| Tocopherol | | | | | | |
| Iron Oxides (CI 77491) (and) | Persea Gratissima | Hydrogenated Vegetable | Yes | APAC | UNIPURE RED | Sensient |
| Persea Gratissima (Avocado) | (Avocado) Oil | Oil (and) Tocopherol | | | LC389 BA | |
| Oil (and) Hydrogenated | | | | | | |
| Vegetable Oil (and) | | | | | | |
| Tocopherol | | | | | | |
| Iron Oxides (CI 77491) (and) | Cocos Nucifera (Coconut) | Aloe Barbadensis Leaf | Yes | APAC | UNIPURE RED | Sensient |
| Cocos Nucifera (Coconut) Oil | Oil | Extract | | | LC389 ALOE | |
| (and) Aloe Barbadensis Leaf | | | | | | |
| Extract | | | | | | |
| Iron Oxides (CI77491) (and) | Phytic Acid | Sodium Hydroxide | Yes | APAC | UNIPURE RED | Sensient |
| Phytic Acid (and) Sodium | | | | | LC389 PHY | |
| Hydroxide | | | | | | |
| Iron Oxides (CI77492) (and) | Phytic Acid | Sodium Hydroxide | Yes | APAC | UNIPURE | Sensient |
| Phytic Acid (and) Sodium | | | | | YELLOW LC186 | |
| Hydroxide | | | | | PHY | |
| Iron Oxides (CI77491) (and) | Hydrogenated Lecithin | | Yes | Global | UNIPURE RED | Sensient |
| Iron Oxides (CI 77499) (and) | | | | | LC381 HLC | |
| Hydrogenated Lecithin | | | | | | |
| Iron Oxides (and) Stearyl | Stearyl Triethoxysilane | | No | Global | Gelest Red Iron | Gelest |
| Triethoxysilane | | | | | Oxide SS | |
| Titanium Dioxide (and) | Stearyl Triethoxysilane | | No | Global | Gelest Titanium | Gelest |
| Stearyl Triethoxysilane | | | | | Dioxide SS | |
| Iron Oxides (and) Disodium | Disodium Carboxyethyl | | No | Global | Red Iron | Gelest |
| Carboxyethyl Siliconate | Siliconate | | | | Oxide HS | |
| Titanium Dioxide (and) | Disodium Carboxyethyl | | No | Global | Gelest Titanium | Gelest |
| Disodium Carboxyethyl | Siliconate | | | | Dioxide HS | |
| Siliconate | | | | | | |
| Iron Oxides (and) | Dimethicone PEG-3 | | No | Global | Gelest Red Iron | Gelest |
| Dimethicone PEG-3 Laurate | Laurate | | | | Oxide ML | |
| Iron Oxides (and) | Trimethylsiloxysilicate | | No | Global | Gelest Red Iron | Gelest |
| Trimethylsiloxysilicate | | | | | Oxide SR | |
| Iron Oxides (and) | Triethoxysilylpropyl | | No | Global | Gelest Red Iron | Gelest |
| Triethoxysilylpropyl Acetyl | Acetyl Hydroxyprolinate | | | | Oxide SF | |
| Hydroxyprolinate | | | | | | |
| Iron Oxides (CI 77491) (and) | Triethoxysilylethyl | | No | Global | KTP-09R | ShinEtsu |
| Triethoxysilylethyl | Polydimethylsiloxyethyl | | | | | |
| Polydimethylsiloxyethyl | Hexyl Dimethicone | | | | | |
| Hexyl Dimethicone | | | | | | |
| Titanium Dioxide (and) | Triethoxysilylethyl | | No | Global | KTP-09W | ShinEtsu |
| Aluminum Hydroxide (and) | Polydimethylsiloxyethyl | | | | | |
| Triethoxysilylethyl | Hexyl Dimethicone | | | | | |
| Polydimethylsiloxyethyl | | | | | | |
| Hexyl Dimethicone | | | | | | |

### 3. Dyes

TABLE 3
Dyes Used in Beauty and Personal Care

| | Colour | | | | | Oil | |
|---|---|---|---|---|---|---|---|
| | Index | Max | Restricted | Regions | | Soluble/Water | |
| INCI | (CI) | % | Use Area | Allowed | Trade Name | Soluble | Supplier |
| Green 6 | 61565 | N/A | External use | Global | Unicert Green | Oil | Sensient |
| | | | only (US) | | K7016-J | | Beauty |
| | | | Hair (CN) | | | | |
| Violet 2 | 60725 | N/A | External use | Global | Unicert Violet | Oil | Sensient |
| | | | only (US) | | K7014-J | | Beauty |
| | | | Hair (CN) | | | | |
| Red 17 | 26100 | N/A | External use | Global | D&C RED 17 | Oil | Sensient |
| | | | only (US, | except CN | K7007 | | Beauty |
| | | | EU, | | | | |
| | | | ASEAN) | | | | |
| Orange | 15510 | N/A | External use | Global | SunCROMA ® | Water | Sun |
| 4 | | | only (US) | | D&C Orange 4 | | Chemical |
| | | | Eye (EU, | | C76-A208 | | |
| | | | CN, | | | | |
| | | | ASEAN) | | | | |
| Red 28 | 45410 | N/A | Eye (US) | Global | SunCROMA ® | Water | Sun |
| Lake | | | | | D&C Red 28 | | Chemical |
| | | | | | C14-A238 | | |
| Red 33 | 17200 | 3% | Eye, Lip 3% | Global | SunCROMA ® | Water | Sun |
| | | | (US) | | D&C Red 33 | | Chemical |
| | | | | | C17-A201 | | |
| Yellow | 47005 | N/A | Eye (US) | Global | SunCROMA ® | Water | Sun |
| 10 | | | | except EU | D&C Yellow 10 | | Chemical |
| | | | | & CN | C71-A213 | | |
| Blue 1 | 42090 | N/A | N/A | Global | SunCROMA ® | Water | Sun |
| | | | | | FD&C Blue 1 | | Chemical |
| | | | | | C39-A220 | | |
| Yellow | 45350 | 6% | External use | Global | SunCROMA ® | Water | Sun |
| 8 | | | only (US) | | D&C Yellow 8 | | Chemical |
| | | | 6% (EU, | | C77-A210 | | |
| | | | ASEAN, | | | | |
| | | | Brazil, CN) | | | | |
| | | | Hair (CN) | | | | |

### 4. Pearls

TABLE 4
Pearls Used in Beauty and Personal Care

| | | Particle | | | | |
|---|---|---|---|---|---|---|
| | | Size | | | | |
| | | D50 | Restricted | Regions | | |
| INCI | Base | (μm) | Use Area | Allowed | Trade Name | Supplier |
| Synthetic | Synthetic | 20-25 | | | SynCrystal | Eckart |
| Fluorphlogopite | Flurophlogopite | | | | Rose Opal | |
| (and) Titanium | | | | | | |
| Dioxide (and) Iron | | | | | | |
| Oxides (and) Tin | | | | | | |
| Oxide | | | | | | |
| Iron Oxides (and) | Synthetic | 20-23 | | | SynCrystal | Eckart |
| Synthetic | Flurophlogopite | | | | Garnet | |
| Fluorphlogopite | | | | | | |
| Synthetic | Synthetic | 20-25 | | | SynCrystal | Eckart |
| Fluorphlogopite | Flurophlogopite | | | | Turquoise | |
| (and) Titanium | | | | | | |
| Dioxide (and) Tin | | | | | | |
| Oxide | | | | | | |
| Synthetic | Synthetic | 20-25 | | | SynCrystal | Eckart |
| Fluorphlogopite | Flurophlogopite | | | | Supernova | |
| (and) Iron Oxides | | | | | Red | |
| (and) Tin Oxide | | | | | | |
| Synthetic | Synthetic | 54-68 | | | SYNCRYSTAL | Eckart |
| Fluorphlogopite | Flurophlogopite | | | | Sparkling | |
| (and) Titanium | | | | | Silver | |
| Dioxide (and) Tin | | | | | | |
| Oxide | | | | | | |
| Calcium Sodium | Calcium Sodium | 23-35 | | | MIRAGE | Eckart |
| Borosilicate (and) | Borosilicate | | | | Bright | |
| Titanium Dioxide | | | | | Luminous | |
| (and) Tin Oxide | | | | | Red | |
| Calcium Sodium | Calcium Sodium | 50-60 | | | MIRAGE | Eckart |
| Borosilicate (and) | Borosilicate | | | | Sparkling | |
| Iron Oxides (and) | | | | | Champagne | |
| Tin Oxide | | | | | | |
| Calcium Sodium | Calcium Sodium | 70-90 | | | MIRAGE | Eckart |
| Borosilicate (and) | Borosilicate | | | | Glamour | |
| Titanium Dioxide | | | | | Silver | |
| (and) Tin Oxide | | | | | | |
| Mica (and) | Mica | 5-25 | | Global | KTZ ® FINE | KOBO |
| Titanium Dioxide | | | | | WHITE | |
| Mica (and) | Mica | 10-60 | | Global | KTZ ® | KOBO |
| Titanium Dioxide | | | | | CLASSIC | |
| | | | | | WHITE | |
| Titanium Dioxide | Mica | 1-15 | | Global | KTZ ® | KOBO |
| (and) Mica | | | | | EXTRAFINE | |
| | | | | | RED | |
| Mica (And) | Mica | 10-125 | | Global | KTZ ® SHIMMER | KOBO |
| Titanium Dioxide | | | | | GREEN | |
| Titanium Dioxide | Mica | 10-60 | | Global | KTZ ® | KOBO |
| (And) Mica (And) | | | | | COLORSET | |
| Carmine | | | | | RedGold | |
| Titanium Dioxide | Mica | 10-60 | Lip | Global | KTZ ® | KOBO |
| (And) Mica (And) | | | | | COLORSET | |
| Ferric | | | | | BlueGold | |
| Ferrocyanide | | | | | | |
| Mica (And) | Mica | 20-200 | | Global | KTZ ® | KOBO |
| Titanium Dioxide | | | | | SUNBURST | |
| (And) Iron Oxides | | | | | GOLD | |
| (CI 77491) | | | | | | |
| Rayon (and) | Rayon | | | Global | Cosmetic | Blue Sun |
| Glycerin (and) | | | | | Bio-Glitter ® | International |
| Aqua (and) Urea | | | | | SPARKLE - | |
| (and) | | | | | Dark Rose | |
| Styrene/Acrylates | | | | | 8345/006H | |
| Copolymer (and) | | | | | | |
| Blue 1 Lake (and) | | | | | | |
| Pigment Red 57:1 | | | | | | |
| (and) CI 77000 | | | | | | |
| Synthetic | Synthetic | 200-260 | | Global | Blondiee ® | CQV |
| Fluorphlogopite | Flurophlogopite | | | | Ultra Sparkle | |
| (and) Iron Oxides | | | | | Gold | |
| (and) Titanium | | | | | | |
| Dioxide | | | | | | |

Various aspects of the disclosure are described in greater detail below.

### II. Compositions of Terpene Polymers

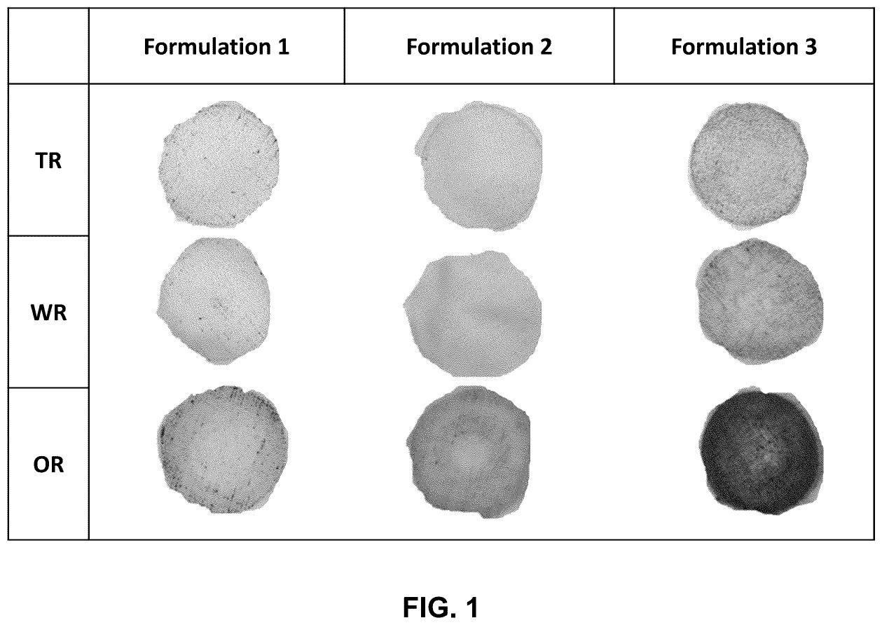

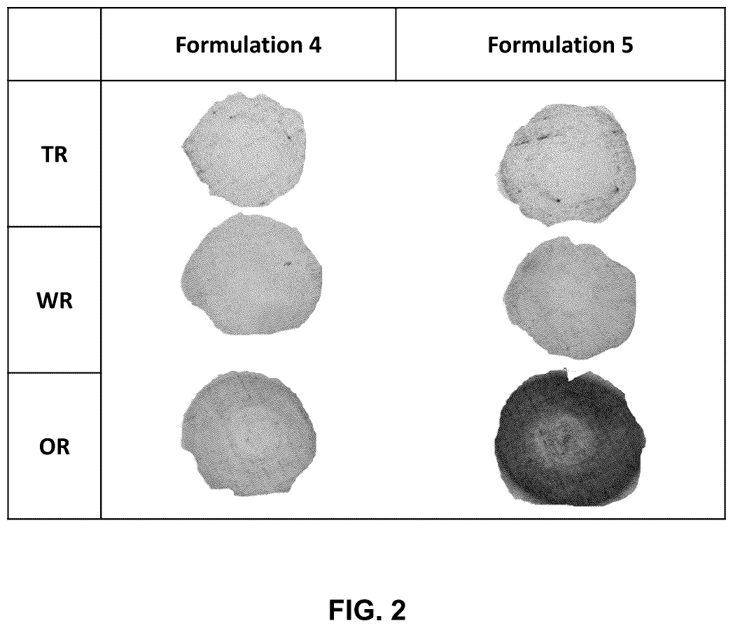

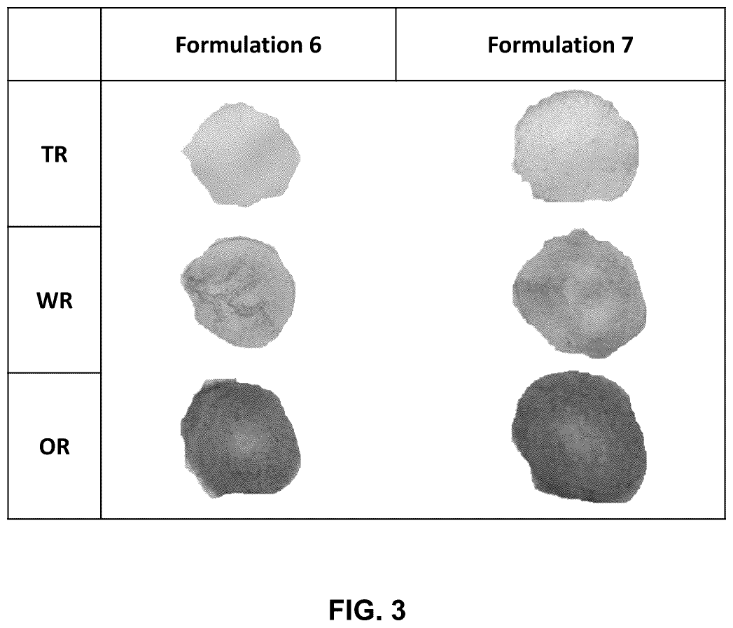

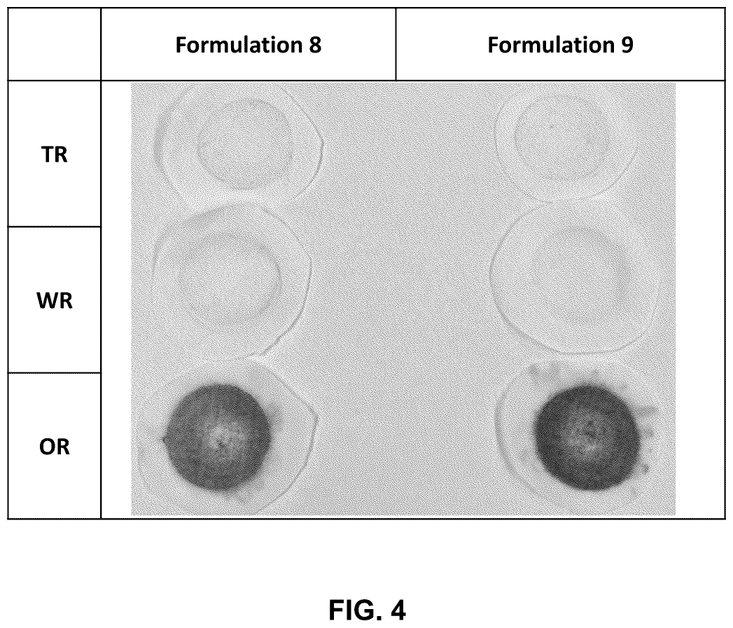

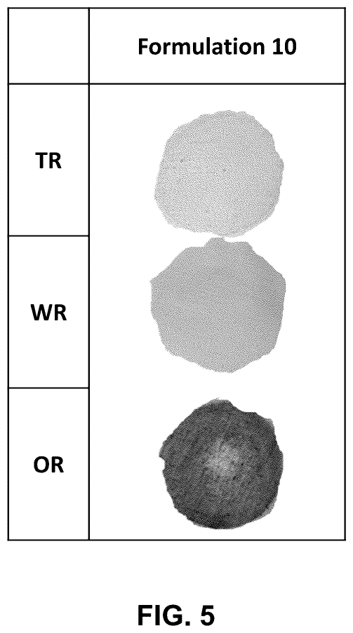

The present disclosure provides a composition (e.g., a personal care composition) comprising at least one terpene polymer with a weight average molecular weight greater than 3,000 g/mol. The composition (e.g., personal care composition) is capable of forming a film on a keratinous substrate, wherein the film is capable of remaining on the keratinous substrate without flaking or transferring to an object that comes in contact with the film. As shown in FIGS. 1-5, Formulation 2 (comprising poly(β-pinene) with a M_{W}=˜8,291 g/mol) and Formulation 4 (comprising poly(β-pinene) with a M_{W}=˜5,739 g/mol) showed good transfer resistance, water resistance, and food oil resistance when compared to the blank (Formulation 3). In particular, the transfer resistance test, water resistance test, and food oil resistance test for Formulation 2 showed very little transference. The transfer resistance test and water resistance test for Formulation 5 (comprising poly(β-pinene) with a M_{W}=˜2,641 g/mol) showed very little transference; however, the increased transfer to a cotton cloth observed with the food oil resistance test indicates that compositions having a molecular weight >2,641 g/mol show greater adherence to a keratinous substrate (e.g., skin, lips, hair, nails) than lower molecular weight compositions.

Additionally, these terpene polymers are not only derived from plant-based monomers but they are also soluble in volatile oils such as isododecane and isoparaffin which are typically used in personal care compositions. Isododecane is well known for its practically weightless feel on the skin and excellent spreadability. Isododecane is also known to provide moisturization to the skin and to condition skin and hair.

In some aspects, the composition (e.g., personal care composition) can comprise one terpene polymer. In some aspects, the composition (e.g., personal care composition) can comprise two terpene polymers. In some aspects, the composition (e.g., personal care composition) can comprise three terpene polymers. In some aspects, the composition (e.g, personal care composition) can comprise four terpene polymers.

In some aspects, the at least one terpene polymer is obtained by polymerizing at least one terpene monomer. In some aspects, the at least one terpene polymer is obtained by polymerizing one terpene monomer. In some aspects, the at least one terpene polymer is obtained by polymerizing two terpene monomers. In some aspects, the at least one terpene polymer is obtained by polymerizing three terpene monomers.

In some aspects, the at least one terpene monomer is selected from the group consisting of a monoterpene, a sesquiterpene, a diterpene, a sesterterpene, a triterpene, a sesquaterpene, a tetraterpene, and combinations thereof.

In some aspects, the at least one terpene polymer is obtained by polymerizing at least one monoterpene monomer. In some aspects, the at least one terpene polymer is obtained by polymerizing one monoterpene monomer. In some aspects, the at least one terpene polymer is obtained by polymerizing two monoterpene monomers. In some aspects, the at least one terpene polymer is obtained by polymerizing three monoterpene monomers.

In some aspects, the at least one terpene monomer is a monoterpene selected from the group consisting of myrcene, ocimene, alloocimene, L-limonene, D-limonene, α-pinene, β-pinene, and combinations thereof.

In some aspects, the at least one terpene polymer is a homopolymer obtained by polymerizing α-pinene, β-pinene, L-limonene, or D-limonene. In some aspects, the at least one terpene polymer is a homopolymer obtained by polymerizing β-pinene.

In some aspects, the at least one terpene polymer is a homopolymer obtained by polymerizing myrcene monomers. In some aspects, the at least one terpene polymer is obtained by polymerizing ocimene monomers. In some aspects, the at least one terpene polymer is obtained by polymerizing alloocimene monomers. In some aspects, the at least one terpene polymer is obtained by polymerizing L-limonene monomers. In some aspects, the at least one terpene polymer is obtained by polymerizing D-limonene monomers. In some aspects, the at least one terpene polymer is obtained by polymerizing α-pinene monomers. In some aspects, the at least one terpene polymer is obtained by polymerizing β-pinene monomers.

In some aspects, the at least one terpene polymer is a copolymer obtained by polymerizing α-pinene, β-pinene, L-limonene, D-limonene, or combinations thereof.

In some aspects, the at least one terpene polymer is a copolymer obtained by polymerizing α-pinene monomers and β-pinene monomers. In some aspects, the at least one terpene polymer is a copolymer obtained by polymerizing α-pinene monomers and L-limonene monomers. In some aspects, the at least one terpene polymer is a copolymer obtained by polymerizing α-pinene monomers and D-limonene monomers. In some aspects, the at least one terpene polymer is a copolymer obtained by polymerizing β-pinene monomers and D-limonene monomers. In some aspects, the at least one terpene polymer is a copolymer obtained by polymerizing β-pinene monomers and L-limonene monomers. In some aspects, the at least one terpene polymer is a copolymer obtained by polymerizing D-limonene and L-limonene monomers.

In some aspects, at least one terpene monomer comprises at least 95% of the total weight of monomers used to obtain the copolymer. In some aspects, at least one terpene monomer comprises at least 50% of the total weight of monomers used to obtain the copolymer. In some aspects, at least one terpene monomer comprises at least 95%, at least 90%, at least 85%, at least 80%, at least 75%, at least 70%, at least 65%, at least 60%, at least 55%, or at least 50% of the total weight of monomers used to obtain the copolymer.

In some aspects, β-pinene comprises at least 95% of the total weight of monomers used to obtain the copolymer. In some aspects, β-pinene comprises at least 50% of the total weight of monomers used to obtain the copolymer. In some aspects, β-pinene comprises at least 95%, at least 90%, at least 85%, at least 80%, at least 75%, at least 70%, at least 65%, at least 60%, at least 55%, or at least 50% of the total weight of monomers used to obtain the copolymer.

In some aspects, the at least one terpene polymer is a homopolymer obtained by hydrogenating at least one terpene polymer. In some aspects, the at least one terpene polymer is a homopolymer obtained by hydrogenating a terpene polymer selected from the group consisting of poly(myrcene), poly(ocimene), poly(alloocimene), poly(L-limonene), poly(D-limonene), poly(α-pinene), and poly(β-pinene). In some aspects, the at least one terpene polymer is a homopolymer obtained by hydrogenating poly(α-pinene). In some aspects, the at least one terpene polymer is a homopolymer obtained by hydrogenating poly(β-pinene). In some aspects, the at least one terpene polymer is a homopolymer obtained by hydrogenating poly(limonene). In some aspects, the at least one terpene polymer is a homopolymer obtained by hydrogenating poly(D-limonene). In some aspects, the at least one terpene polymer is a homopolymer obtained by hydrogenating poly(L-limonene).

In some aspects, the at least one terpene polymer is hydrogenated from about 0.1% to 100%. In some aspects, the at least one terpene polymer is hydrogenated from about 0.1% to about 100%, from about 0.1% to about 99%, from about 0.1% to about 90%, from about 0.1% to about 75%, from about 0.1% to about 50%, from about 0.1% to about 25%, from about 0.1% to about 10%, from about 0.1% to about 1%, from about 1% to 100%, from about 1% to about 99%, from about 1% to about 90%, from about 1% to about 75%, from about 1% to about 50%, from about 1% to about 25%, from about 1% to about 10%, from about 10% to 100%, from about 10% to about 99%, from about 10% to about 90%, from about 10% to about 75%, from about 10% to about 50%, from about 10% to about 25%, from about 25% to 100%, from about 25% to about 99%, from about 25% to about 90%, from about 25% to about 75%, from about 25% to about 50%, from about 50% to 100%, from about 50% to about 99%, from about 50% to about 90%, from about 50% to about 750%, from about 750% to 100%, from about 750% to about 99%, from about 75% to about 9000, from about 900% to 10000, from about 900% to about 9900, or from about 9900 to 100%.

In some aspects, the at least one terpene polymer is a copolymer obtained by hydrogenating a copolymer of at least two terpene monomers. In some aspects, the at least one terpene polymer is obtained by hydrogenating a copolymer of at least one terpene monomer and at least one monomer that is not a terpene.

In some aspects, the at least one terpene polymer is a copolymer further comprising at least one monomer that is not a terpene.

In some aspects, the at least one monomer that is not a terpene is selected from the group consisting of an acrylate, an anhydride, a styrene, an ethylene, a butadiene, a urethane, a propylene, an imidazole, a silicone, a vinyl pyridine, a carbonate, and combinations thereof.

In some aspects, the weight average molecular weight (M_{W}) of the at least one terpene polymer is greater than or equal to about 3,000 g/mol. In some aspects, the weight average molecular weight of the at least one terpene polymer is from about 3,000 g/mol to about 2,000,000 g/mol. In some aspects, the weight average molecular weight of the at least one terpene polymer is from about 5,000 g/mol to about 50,000 g/mol. In some aspects, the weight average molecular weight of the at least one terpene polymer is from about 3,500 g/mol to about 10,000 g/mol. In some aspects, the weight average molecular weight of the at least one terpene polymer is from about 3,000 g/mol to about 10,000 g/mol. In some aspects, the weight average molecular weight of the at least one terpene polymer is from about 3,000 g/mol to about 2,000,000 g/mol, from about 3,000 g/mol to about 1,000,000 g/mol, from about 3,000 g/mol to about 500,000 g/mol, from about 3,000 g/mol to about 100,000 g/mol, from about 3,000 g/mol to about 20,000 g/mol, from about 3,000 g/mol to about 10,000 g/mol, from about 3,000 g/mol to about 5,000 g/mol, from about 3,000 g/mol to about 3,500 g/mol, from about 3,500 g/mol to about 2,000,000 g/mol, from about 3,500 g/mol to about 1,000,000 g/mol, from about 3,500 g/mol to about 500,000 g/mol, from about 3,500 g/mol to about 100,000 g/mol, from about 3,500 g/mol to about 20,000 g/mol, from about 3,500 g/mol to about 10,000 g/mol, from about 3,500 g/mol to about 5,000 g/mol, from about 5,000 g/mol to about 2,000,000 g/mol, from about 5,000 g/mol to about 1,000,000 g/mol, from about 5,000 g/mol to about 500,000 g/mol, from about 5,000 g/mol to about 100,000 g/mol, from about 5,000 g/mol to about 20,000 g/mol, from about 5,000 g/mol to about 10,000 g/mol, from about 10,000 g/mol to about 2,000,000 g/mol, from about 10,000 g/mol to about 1,000,000 g/mol, from about 10,000 g/mol to about 500,000 g/mol, from about 10,000 g/mol to about 100,000 g/mol, from about 10,000 g/mol to about 20,000 g/mol, from about 20,000 g/mol to about 2,000,000 g/mol, from about 20,000 g/mol to about 1,000,000 g/mol, from about 20,000 g/mol to about 500,000 g/mol, from about 20,000 g/mol to about 100,000 g/mol, from about 100,000 g/mol to about 2,000,000 g/mol, from about 100,000 g/mol to about 1,000,000 g/mol, from about 100,000 g/mol to about 500,000 g/mol, from about 500,000 g/mol to about 2,000,000 g/mol, from about 500,000 g/mol to about 1,000,000 g/mol, or from about 1,000,000 g/mol to about 2,000,000 g/mol.

In some aspects, the weight average molecular weight of the at least one terpene polymer is about 3,000 g/mol, about 3,500 g/mol, about 5,000 g/mol, about 10,000 g/mol, about 25,000 g/mol, about 50,000 g/mol, about 75,000 g/mol, about 100,000 g/mol, about 250,000 g/mol, about 500,000 g/mol, about 750,000 g/mol, about 1,000,000 g/mol, about 1,250,000 g/mol, about 1,500,000 g/mol, 1,750,000 g/mol, or about 2,000,000 g/mol. In some aspects, the weight average molecular weight of the at least one terpene polymer is about 20,000 g/mol. In some aspects, the weight average molecular weight of the at least one terpene polymer is about 10,000 g/mol. In some aspects, the weight average molecular weight of the at least one terpene polymer is about 8,200 g/mol. In some embodiments, the weight average molecular weight of the at least one terpene polymer is about 8,291 g/mol. The weight average molecular weight is measured by gel permeation chromatography.

The softening point of a terpene polymer is the temperature at which the polymer flows. Softening point can be measured by Differential Scanning Calorimetry (DSC) using a DSC 250 (TA Instruments, New Castle, DE). The onset of the glass transition is indicated by the softening of the polymer and ends once the polymer is completely free-flowing. Softening point is assigned to the temperature at the peak of the heat flow curve obtained by DSC.

In some aspects, the terpene polymer can have a glass transition temperature of greater than or equal to about 50° C. In some aspects, the terpene polymer can have a glass transition temperature of greater than or equal to about 80° C. In some aspects, the terpene polymer can have a glass transition temperature of from about 50° C. to about 300° C., from about 50° to about 200° C., from about 50° C. to about 100° C., from about 50° C. to about 80° C., from about 80° C. to about 300° C., from about 80° C. to about 200° C., from about 80° C. to about 100° C., from about 100° C. to about 300° C., from about 100° C. to about 200° C., or from about 200° C. to about 300° C.

In some aspects, the weight average molecular weight of the at least one terpene polymer, wherein the at least one terpene polymer is β-pinene, is greater than or equal to about 3,000 g/mol. In some aspects, the weight average molecular weight of the at least one terpene polymer, wherein the at least one terpene polymer is β-pinene, is from about 3,000 g/mol to about 2,000,000 g/mol. In some aspects, the weight average molecular weight of the at least one terpene polymer, wherein the at least one terpene polymer is β-pinene, is from about 5,000 g/mol to about 50,000 g/mol. In some aspects, the weight average molecular weight of the at least one terpene polymer, wherein the at least one terpene polymer is β-pinene, is from about 3,500 g/mol to about 10,000 g/mol. In some aspects, the weight average molecular weight of the at least one terpene polymer, wherein the at least one terpene polymer is β-pinene, is from about 3,000 g/mol to about 10,000 g/mol. In some aspects, the weight average molecular weight of the at least one terpene polymer, wherein the at least one terpene is β-pinene, is from about 3,000 g/mol to about 2,000,000 g/mol, from about 3,000 g/mol to about 1,000,000 g/mol, from about 3,000 g/mol to about 500,000 g/mol, from about 3,000 g/mol to about 100,000 g/mol, from about 3,000 g/mol to about 20,000 g/mol, from about 3,000 g/mol to about 10,000 g/mol, from about 3,000 g/mol to about 5,000 g/mol, from about 3,000 g/mol to about 3,500 g/mol, from about 3,500 g/mol to about 2,000,000 g/mol, from about 3,500 g/mol to about 1,000,000 g/mol, from about 3,500 g/mol to about 500,000 g/mol, from about 3,500 g/mol to about 100,000 g/mol, from about 3,500 g/mol to about 20,000 g/mol, from about 3,500 g/mol to about 10,000 g/mol, from about 3,500 g/mol to about 5,000 g/mol, from about 5,000 g/mol to about 2,000,000 g/mol, from about 5,000 g/mol to about 1,000,000 g/mol, from about 5,000 g/mol to about 500,000 g/mol, from about 5,000 g/mol to about 100,000 g/mol, from about 5,000 g/mol to about 20,000 g/mol, from about 5,000 g/mol to about 10,000 g/mol, from about 10,000 g/mol to about 2,000,000 g/mol, from about 10,000 g/mol to about 1,000,000 g/mol, from about 10,000 g/mol to about 500,000 g/mol, from about 10,000 g/mol to about 100,000 g/mol, from about 10,000 g/mol to about 20,000 g/mol, from about 20,000 g/mol to about 2,000,000 g/mol, from about 20,000 g/mol to about 1,000,000 g/mol, from about 20,000 g/mol to about 500,000 g/mol, from about 20,000 g/mol to about 100,000 g/mol, from about 100,000 g/mol to about 2,000,000 g/mol, from about 100,000 g/mol to about 1,000,000 g/mol, from about 100,000 g/mol to about 500,000 g/mol, from about 500,000 g/mol to about 2,000,000 g/mol, from about 500,000 g/mol to about 1,000,000 g/mol, or from about 1,000,000 g/mol to about 2,000,000 g/mol.

In some aspects, the present disclosure is directed to a composition (e.g., a personal care composition) comprising at least one terpene polymer, wherein the terpene polymer is β-pinene with a weight average molecular weight greater than 3,000 g/mol. In some aspects, the terpene polymer is β-pinene with a weight average molecular weight greater than 5,000 g/mol. In some aspects, the terpene polymer is β-pinene with a weight average molecular weight greater than 8,000 g/mol. In some aspects, the terpene polymer is β-pinene with a weight average molecular weight of about 8,291 g/mol.

In some aspects, the weight of the at least one terpene polymer in the composition (e.g., personal care composition) is from about 0.1% to about 30% of the total weight of the composition. In some aspects, the weight of the at least one terpene polymer in the composition (e.g., a personal care composition) is from about 0.1% to about 30%, from about 0.1% to about 25%, from about 0.1% to about 20%, from about 0.1% to about 15%, from about 0.1% to about 10%, from about 0.1% to about 5%, from about 0.1% to about 1%, from about 1% to about 30%, from about 1% to about 25%, from about 1% to about 20%, from about 1% to about 15%, from about 1% to about 10%, from about 1% to about 5%, from about 5% to about 30%, from about 5% to about 25%, from about 5% to about 20%, from about 5% to about 15%, from about 5% to about 10%, from about 10% to about 30%, from about 10% to about 25%, from about 10% to about 20%, from about 10% to about 15%, from about 15% to about 30%, from about 15% to about 25%, from about 15% to about 20%, from about 20% to about 30%, from about 20% to about 25%, or from about 25% to about 30% of the total weight of the composition.

In some aspects, the present disclosure is directed to a composition (e.g., a personal care composition) comprising at least one terpene polymer and further comprising:
(a) at least one non-volatile oil; or
(b) at least one volatile oil; or
(c) at least one elastomer; or
(d) a combination thereof.

In some aspects, the composition (e.g., a personal care composition) can comprise at least one terpene polymer and at least one non-volatile oil. In some aspects, the composition (e.g., a personal care composition) can comprise at least one terpene polymer and at least one volatile oil. In some aspects, the composition (e.g., a personal care composition) can comprise at least one terpene polymer and at least one elastomer. In some aspects, the composition (e.g., a personal care composition) can comprise as least one terpene polymer, at least one non-volatile oil, and at least one elastomer. In some aspects, the composition (e.g., a personal care composition) can comprise at least one terpene polymer, at least volatile oil, and at least one elastomer. In some aspects, the composition (e.g., a personal care composition) can comprise at least one terpene polymer, at least one non-volatile oil, at least one volatile oil, and at least one elastomer.

In some aspects, the composition (e.g., a personal care composition) can comprise at least one terpene polymer and at least one non-volatile oil. The phrase “non-volatile oil” refers to an oil remaining on a surface, such as skin, at ambient temperature and atmospheric pressure for a plurality of hours, in the absence of friction, and/or having a vapor pressure less than 0.001 mm Hg under these conditions. In some aspects, the non-volatile oil is a mineral or synthetic branched hydrocarbon, a C₅-C₂₄ acid, a C₈-C₂₄ alcohol or polyol, a C₆-C₂₀ fatty acid triglyceride, a vegetable oil, a dialkyl carbonate, a branched and/or unsaturated fatty acid, a branched and/or unsaturated fatty alcohol, a silicone oil, a fluorosilicone oil, a fluorinated oil, or a combination thereof. In some embodiments, the non-volatile oil is selected from the group consisting of ethyl myristate, isopropyl myristate, diisopropyl adipate, and other fatty acids. In some aspects, the non-volatile oil is selected from the group consisting of a polyisobutylene, a polyethylene, a silicone oil, a silicone gum, a butter, a lipid, a fatty acid, an alcohol, a ceramide, an ether, a linear or branched hydrocarbon, a linear or branched ester, a vegetable oil, and combinations and/or oligomers thereof. In some aspects, the non-volatile oil is a silicone oil or a silicone gum. In some aspects, the non-volatile oil is a silicone gum.

As used herein, the term “butter” refers to an ingredient that softens and soothes the skin by creating a protective barrier that locks in moisture and prevents water loss.

As used herein, the term “lipid” refers to an organic compound that is insoluble in polar solvents such as water and soluble in organic solvents and that are made up of repeating units called fatty acids which are mostly straight-chain hydrocarbons with a terminal carboxy group.

As used herein, the term “fatty acid” refers to a carboxylic acid with an aliphatic chain, which can be either saturated or unsaturated. Most naturally occurring fatty acids have an unbranched chain with an even number of carbon atoms from 4 to 28.

As used herein, the term “alcohol” refers to a compound having formula R¹OH, wherein R¹ is C₁-alkyl C₂-alkyl, C₃alkyl, C₄-alkyl, C₅-alkyl, C₆-alkyl, C₇-alkyl, C₈-alky C₉-alkyl, C₁₀-alkyl, C₁₁-alkyl, C₁₂-alkyl, C₁₃-alkyl, C₁₄-alkyl, C₁₅-alkyl, C₁₆-alkyl, C₁₇-alkyl, C₁₈-alkyl, C₁₉-alkyl, C₂₀-alkyl, C₂₁-alkyl, C₂₂-alkyl, C₂₃alkyl, or C₂₄-alkyl.

As used herein, the term “C₁-alkyl” refers to methyl.

As used herein, the term “C₂-alkyl” refers to ethyl.

As used herein, the term “C₃-alkyl,” refers to prop-1-yl and prop-2-yl (isopropyl).

As used herein, the term “C₄-alkyl,” refers to but-1-yl, but-2-yl, 2-methylprop-1-yl and 2-methylprop-2-yl (tert-butyl).

As used herein, the term “C₅-alkyl,” refers to 2,2-dimethylprop-1-yl (neo-pentyl), 2-methylbut-1-yl, 2-methylbut-2-yl, 3-methylbut-1-yl, 3-methylbut-2-yl, pent-1-yl, pent-2-yl, and pent-3-yl.

As used herein, the term “C₆-alkyl,” refers to 2,2-dimethylbut-1-yl, 2,3-dimethylbut-1-yl, 2,3-dimethylbut-2-yl, 3,3-dimethylbut-1-yl, 3,3-dimethylbut-2-yl, 2-ethylbut-1-yl, hex-1-yl, hex-2-yl, hex-3-yl, 2-methylpent-1-yl, 2-methylpent-2-yl, 2-methylpent-3-yl, 3-methylpent-1-yl, 3-methylpent-2-yl, 3-methylpent-3-yl, 4-methylpent-1-yl, and 4-methylpent-2-yl.

As used herein, the term “ceramide” refers to a molecule comprising a sphingosine core structure. A sphingosine is an amino alcohol with an unsaturated hydrocarbon chain that is typically 18-carbon or 20-carbon in length, which forms a primary part of sphingolipids (e.g., ceramides). The unsaturated hydrocarbon chain is attached to the amino acid serine to form the sphingosine. The term “ceramide” encompasses natural ceramides and ceramide analogs (e.g., synthetic or natural ceramide analogs). In some embodiments, the ceramide is a sphingolipid composed of sphingosine and a fatty acid. In some embodiments, the ceramide of the present disclosure is a ceramide analog. For example, the ceramide of the present disclosure may contain additional chemical moieties appended to a natural ceramide or contain modifications compared to a natural ceramide. Non-limiting examples of ceramide analogs that may be used in accordance with the present disclosure include 2-hydroxy-ceramide, diene-deoxy-ceramide, dihydroceramide, dihydroceramide phosphate, o-acyl-ceramide, ceramide phosphate, sphinganine, and methyl-sphingosine.

As used herein, the term “ether” refers to an organic compound that contains two hydrocarbon groups linked by an oxygen atom. Non-limiting examples of ethers that may be used in accordance with the present disclosure include diethyl ether, methyl ethyl ether, 2-methoxy-2-methylpropane, and phenoxybenzene.

In some aspects, the composition (e.g., a personal care composition) can comprise at least one non-volatile oil. In some aspects, the composition (e.g., a personal care composition) can comprise at least two non-volatile oils. In some aspects, the composition (e.g., a personal care composition) can comprises at least three non-volatile oils. In some aspects, the composition can comprise one, two, three, four, five, or six non-volatile oils.

In some aspects, the composition (e.g., a personal care composition) can comprise a non-volatile oil in a weight ratio from about 0.01% to about 80% of the total weight of the composition. In some aspects, the composition (e.g., a personal care composition) can comprise a non-volatile oil in a weight ratio from about 0.01% to about 80%, from about 0.01% to about 60%, from about 0.01% to about 40%, from about 0.01% to about 20%, from about 0.01% to about 10%, from about 0.01% to about 5%, from about 0.01% to about 1%, from about 1% to about 80%, from about 1% to about 60%, from about 1% to about 40%, from about 1% to about 20%, from about 1% to about 10%, from about 1% to about 5%, from about 5% to about 80%, from about 5% to about 60%, from about 5% to about 40%, from about 5% to about 20%, from about 5% to about 10%, from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% of the total weight of the composition. In some aspects, the composition can comprise a non-volatile oil in a weight ratio of about 0.01%, about 1%, about 5%, about 10%, about 20%, about 40%, about 60%, or about 80% of the total weight of the composition.

In some aspects, the non-volatile oil is a plasticizer. As used herein, the term “plasticizer” refers to a substance added to a synthetic resin to promote flexibility and the ability of the substance to be shaped or molded and to reduce brittleness. In some aspects, the plasticizer provides additional consistency and stability to the composition. Plasticizers can fix graininess that occurs when solids migrate and agglomerate. Plasticizers can also fix thermal stability issues by increasing melt point, without affecting the overall texture.

In some aspects, the present disclosure is directed to a composition (e.g., a personal care composition) comprising at least one terpene polymer and at least one plasticizer.

In some aspects, the composition (e.g., personal care composition) can comprise at least one plasticizer. In some aspects, the composition (e.g., personal care composition) can comprise at least two plasticizers. In some aspects, the composition (e.g., personal care composition) can comprises at least three plasticizers. In some aspects, the composition (e.g., personal care composition) can comprise one, two, three, four, five, or six plasticizers.

In some aspects, the composition (e.g., personal care composition) can comprise a plasticizer in a weight ratio from about 0.01% to about 80% of the total weight of the composition. In some aspects, the composition (e.g., personal care composition) comprises a plasticizer in a weight ratio from about 0.01% to about 80%, from about 0.01% to about 60%, from about 0.01% to about 40%, from about 0.01% to about 20%, from about 0.01% to about 10%, from about 0.01% to about 5%, from about 0.01% to about 1%, from about 1% to about 80%, from about 1% to about 60%, from about 1% to about 40%, from about 1% to about 20%, from about 1% to about 10%, from about 1% to about 5%, from about 5% to about 80%, from about 5% to about 60%, from about 5% to about 40%, from about 5% to about 20%, from about 5% to about 10%, from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% of the total weight of the composition. In some aspects, the compositions comprise a plasticizer in a weight ratio of about 0.01%, about 1%, about 5%, about 10%, about 20%, about 40%, about 60%, or about 80% of the total weight of the composition.

In some aspects, the plasticizer is selected from the group consisting of cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂₋₁₃ alkyl lactate, C₁₂₋₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂₋₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxystearate, trimethylpentanediol/adipic acid/glycerin crosspolymer, capryloyl glycerin/sebacic acid copolymer, brassica campestris/aleurites fordi oil copolymer, PPG-12/SMDI copolymer, polyisobutylene, polyisobutene 1200, polyhydroxystearic acid, and dicaprylyl/capryl sebacate, and combinations thereof.

In some aspects, the plasticizer is selected from the group consisting of a trimethylpentanediol/adipic acid/glycerin crosspolymer, capryloyl glycerin/sebacic acid copolymer, brassica campestris/aleurites fordi oil copolymer, PPG-12/SMDI copolymer, polyisobutylene, polyisobutene 1200, isocetyl salicylate, polyhydroxystearic acid, dicaprylyl/capryl sebacate, and combinations thereof.

In some aspects, the plasticizer is selected from the group consisting of LEXOREZ 200 MB, VELLAPLEX MB, GLOSSAMER L6600, ADEKA NOL OU-1, LIPFEEL Natural MB, polyisobutylene, polyisobutene 1200, DERMOL ICSA, KESTER WAX K-60P, HALLGREEN CCS, and combinations thereof.

In some aspects, the composition (e.g., personal care composition) can comprise at least one terpene polymer and at least one volatile oil. The phrase “volatile oil” refers to an oil capable of evaporating on a keratin material such as the skin, the lips, or the hair in less than one hour at ambient temperature (25° C.), at a vapor pressure of greater than or equal to 26.66 Pa, and at atmospheric pressure (760 mm Hg).

In some aspects, the composition (e.g., personal care composition) can comprise at least one volatile oil. In some aspects, the composition (e.g., personal care composition) can comprise at least two volatile oils. In some aspects, the composition (e.g., personal care composition) can comprises at least three volatile oils. In some aspects, the composition can comprise one, two, three, four, five, or six volatile oils.

In some aspects, the volatile oil is a silicone oil such as decamethylcyclopentasiloxane, polymethylcyclohexasiloxane, and polymethylcyclotetrasiloxane; a linear, branched, or mixture of hydrocarbon oils such as nonane, decane, dodecane, isododecane, isohexadecane, isoparaffin (C₁₀-C₁₄), C₉-C₁₂ alkane, or a combination thereof. In some aspects, the volatile oil is isododecane.

In some aspects, the present disclosure is directed to a composition (e.g., personal care composition) that can comprise at least one volatile alcohol. In some aspects, the present disclosure is directed to a composition (e.g., personal care composition) that does not comprise a volatile alcohol. The term “volatile alcohol” refers to a compound containing at least one hydroxyl group and in which more than 95% by weight of the compound can evaporate in less than one hour at ambient temperature (25° C.) and atmospheric pressure (760 mm Hg) on contact with a keratin material such as the skin or the hair. In some aspects, the volatile alcohol is a lower C₁-C₅ alcohol such as methanol, ethanol, propanol, isopropanol, n-butanol, tert-butanol, and combinations thereof.

In some aspects, the present disclosure is directed to a composition (e.g., a personal care composition) comprising at least one terpene polymer and at least one plasticizer.

In some aspects, the composition is a personal care composition. The phrase “personal care composition” or “personal care formulation” is used to describe a chemical composition used for the purpose of cleansing, conditioning, grooming, beautifying, or otherwise enhancing the appearance of the human body.

In some aspects, the present disclosure is directed to a composition (e.g., a personal care composition) comprising at least one terpene polymer, a plasticizer, and an elastomer. Elastomers, commonly described as rubbery materials, differ from plasticizers in that they have a great capacity for large elastic deformation under an applied stress. As used herein, the term “elastomer” refers to a natural or synthetic polymer having the ability to resume its normal shape spontaneously after contraction, dilatation, or distortion.

In some aspects, the composition (e.g., personal care composition) comprises at least one terpene and an elastomer.

In some aspects, the elastomer is an elastomer gel from epoxidized vegetable oil. U.S. Appl. Publication No. 2022/0194912, incorporated herein by reference in its entirety, describes the preparation of an elastomer comprising the reaction product of at least one epoxidized molecule and at least one crosslinker.

In some aspects, the elastomer comprises the reaction product of
(a) at least one compound of formula (I)

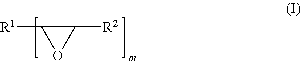

wherein
R¹ is C₆-C_{100,000} alkyl group, C₆-C_{100,000} heteroalkyl group, C₆-C_{100,000} alkene group, C₆-C_{100,000} heteroalkene group, C₆-C_{100,000} alkyne group, C₆-C_{100,000} heteroalkyne group, C₆-C_{100,000} cyclic group, or C₆-C_{100,000} heterocyclic group;
R² is hydrogen, C₁-C_{100,000} alkyl group, C₁-C_{100,000} heteroalkyl group, C₂-C_{100,000} alkene group, C₂-C_{100,000} heteroalkene group, C₂-C_{100,000} alkyne group, C₂-C_{100,000} heteroalkyne group, C₃-C_{100,000} cyclic group, or C₃-C_{100,000} heterocyclic group; and
   m is an integer from 2 to 1,000; and
(b) at least one crosslinker selected from the group consisting of
(1) a carboxylic acid comprising the structure of formula (II)

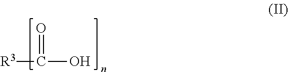

wherein
   R³ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
   n is an integer from 2 to 10;
(2) an anhydride comprising the structure of formula (III)

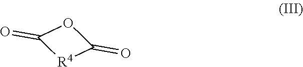

wherein
   R⁴ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
an amine comprising the structure of formula (IV)

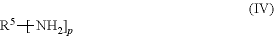

wherein
   R⁵ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
   p is an integer from 2 to 10;
an alcohol comprising the structure of formula (V)

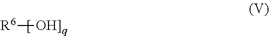

wherein
   R⁶ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
   q is an integer from 2 to 10;
a hydroxyl carboxylic acid comprising the structure of formula (VI)

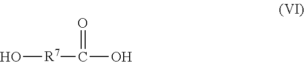

wherein
   R⁷ is C₁-C₂₀₀ alkyl group, C₁-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
an amine carboxylic acid comprising the structure of formula (VII)

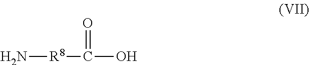

wherein
   R⁸ is C₁-C₂₀₀ alkyl group, C₁-C_{100,000} heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
and combinations thereof.

In some aspects, the elastomer is a crosslinked polyester elastomer. U.S. Appl. Publication No. 2022/0195178, incorporated herein by reference in its entirety, describes the preparation of a crosslinked polyester elastomer comprising the reaction product of (i) at least one polycarboxylic acid, at least one polycarboxylic ester, or a combination thereof, and (ii) at least one polyol, wherein (a) the at least one polycarboxylic acid or at least one polycarboxylic acid ester and the at least one polyol have a total of at least five carboxyl and hydroxyl functional groups and (b) there must be at least three carboxyl or hydroxyl functional groups on the at least one polycarboxylic acid, the at least one polycarboxylic acid ester, or the at least one polyol. In another aspect, the reaction is in the presence of a first low molecular weight organic solvent thereby forming a crosslinking polymer structure. In a further aspect, the gel prepared from the elastomer is used for a personal care formulation. As used herein, the phrase “crosslinked polyester elastomer” refers to a natural or synthetic crosslinked polyester having the ability to resume its normal shape spontaneously after contraction, dilatation, or distortion.

In some aspects, the elastomer comprises the reaction product of
(i) at least one polycarboxylic acid of formula (IA)

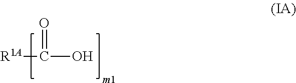

wherein
R^{1A} is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
m1 is an integer from 2 to 10; or
(ii) at least one polycarboxylic acid ester of formula (IIA)

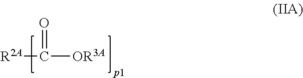

wherein
R^{2A} is C₁-C₂₂ alkyl group, C₂-C₂₂ alkene group, or C₃-C₂₂ cyclic group;
R^{3A} is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
p1 is an integer from 3 to 10; or
(iii) a combination thereof, and
(iv) at least one polyol of formula (IIIA)

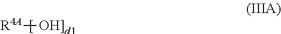

wherein
R^{4A} is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
d1 is an integer from 2 to 10.

In some aspects, the elastomer is a crosslinked polyester elastomer. U.S. Appl. No. 2024/0382405 A1, and U.S. Appl. No. 2024/0027571 A1, each of which is incorporated herein by reference in their entireties, describes the preparation of a crosslinked polyester elastomer comprising the reaction product of (i) at least one di-carboxylic acid or tri-carboxylic acid; (ii) at least one mono-carboxylic acid; and (ii) at least one polyol.

In some aspects, the present disclosure provides a polyester elastomer comprising the reaction product of
(i) at least one di-carboxylic acid or tri-carboxylic acid;
(ii) at least one mono-carboxylic acid; and
(iii) at least one polyol.

In some aspects, the present disclosure provides an elastomer prepared by reacting:
(i) at least one di-carboxylic acid of formula (IB)

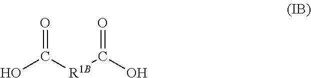

wherein
R^{1B} is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group; or
at least one tri-carboxylic acid of formula (IIB)

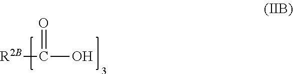

wherein
R^{2B} is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group;
(ii) at least one mono-carboxylic acid of formula (IIIB)

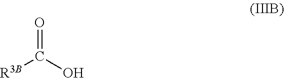

wherein
R^{3B} is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group; and
(iii) at least one polyol of formula (IVB)

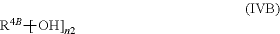

wherein
R^{4B} is C₃-C₅₀ alkyl group, C₃-C₅₀ heteroalkyl group, C₃-C₅₀ alkene group, C₃-C₅₀ heteroalkene group, C₃-C₅₀ cyclic group, or C₃-C₅₀ heterocyclic group; and
n2 is an integer from 2 to 10.

In an aspect, the present disclosure provides a method of preparing a polyester elastomer comprising reacting:
(i) at least one di-carboxylic acid or tri-carboxylic acid;
(ii) at least one mono-carboxylic acid; and
(iii) at least one polyol.

In an aspect, the preparation of polyester elastomer is without solvent or emollient. In an aspect, the preparation of polyester elastomer is with solvent or emollient as defined herein.

In an aspect, the polyester elastomer composition is a polyester elastomer combined with one or more solvents or emollients, which can be converted to a polyester elastomer gel (the swollen polyester elastomer), for example, by applying a shear force to the composition.

In an aspect, the present disclosure provides for the use of a gel, or a powder prepared from a polyester elastomer described herein, in the manufacture of a personal care formulation.

In some aspects, the elastomer comprises the reaction product of
(i) at least one activated polycarboxylic acid is a compound of formula (IC)

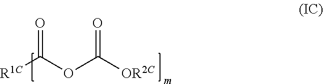

wherein
R^{1C} is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
R^{2C} is a C₁-C₆₀ monovalent hydrocarbon group; and
m is an integer from 2 to 10; and
(ii) the at least one polyol is a compound of formula (IIC)

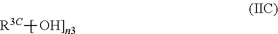

wherein
R^{3C} is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
n2 is an integer from 2 to 10.

In some aspects, the elastomer is a siloxane elastomer. The term “siloxane elastomer” as used herein, refers to a siloxane polymer exhibiting elastomer properties. The term “siloxane polymer” as used herein, refers to an organo-silicone polymer which comprises Si—O—Si linkages as the backbone of the polymer.

In some aspects, the composition (e.g., personal care composition) comprises an elastomer in a weight ratio from about 0.01% to about 80% of the total weight of the composition. In some aspects, the composition (e.g., personal care composition) comprises an elastomer in a weight ratio from about 0.01% to about 80%, from about 0.01% to about 60%, from about 0.01% to about 40%, from about 0.01% to about 20%, from about 0.01% to about 10%, from about 0.01% to about 5%, from about 0.01% to about 1%, from about 1% to about 80%, from about 1% to about 60%, from about 1% to about 40%, from about 1% to about 20%, from about 1% to about 10%, from about 1% to about 5%, from about 5% to about 80%, from about 5% to about 60%, from about 5% to about 40%, from about 5% to about 20%, from about 5% to about 10%, from about 10% to about 80%, from about 10% to about 60%, from about 10% to about 40%, from about 10% to about 20%, from about 20% to about 80%, from about 20% to about 60%, from about 20 to about 40%, from about 400% to about 800%, from about 400% to about 600%, or from about 600% to about 80% of the total weight of the composition. In some aspects, the composition (e.g., personal care composition) comprises an elastomer in a weight ratio of about 0.01%, about 1%, about 5%, about 10%, about 20%, about 40%, about 60%, or about 80% of the total weight of the composition.

The composition (e.g., personal care composition) comprising at least one terpene polymer can further comprise at least one additional component selected from the group consisting of water, an aqueous solvent (e.g. alcohol or other water compatible solvent), a non-aqueous solvent, emollients, humectants, oils (polar and non-polar), conditioning agents, emulsifiers, surfactants, thickeners, rheology modifiers, stiffening agents, medicaments, fragrances, preservatives, deodorant components, anti-perspirant compounds, skin protecting agents, pigments, sunscreens and mixtures thereof, among others, wherein said composition, when deposited onto a surface, dries (after evaporation of solvent and water) into a uniform, flexible, durable film exhibiting water resistance, flexibility, durability and optionally, transfer resistance when the film also comprises a pigment, dye, oil and/or active component.

In some aspects, the additional component(s) comprise from about 0.01% to about 80%, from about 0.01% to about 60%, from about 0.01% to about 40%, from about 0.01% to about 20%, from about 0.01% to about 10%, from about 0.01% to about 5%, from about 0.01% to about 1%, from about 1% to about 80%, from about 1% to about 60%, from about 1% to about 40%, from about 1% to about 20%, from about 1% to about 10%, from about 1% to about 5%, from about 5% to about 80%, from about 5% to about 60%, from about 5% to about 40%, from about 5% to about 20%, from about 5% to about 10%, from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% of the total weight of the composition. In some aspects, the compositions comprise the additional component(s) in a weight ratio of about 0.01%, about 1%, about 5%, about 10%, about 20%, about 40%, about 60%, or about 80% of the total weight of the composition.

In some aspects, the composition is in the form of an emulsion.

As used herein, the term “emulsion” refers to a colloidal dispersion of two or more liquid immiscible phases (or substantially immiscible phases) in the form of droplets. One of the liquid phases is normally a dispersed phase and another one is a continuous phase, wherein the dispersed phase is scattered in the continuous phase as a plurality of droplets. The emulsion can be in a form of a macroemulsion, a microemulsion, or a nanoemulsion based on the size of the droplets. The emulsion is an oil-in-water (o/w) emulsion if the continuous phase is an aqueous solution or a water-in-oil (w/o)-type if the continuous phase is an oil. Other examples of emulsions include oil-in-water-in-oil (o/w/o) emulsions, which comprise oil droplets contained within aqueous droplets dispersed in a continuous oil phase. As used herein the term “oil” refers to any nonpolar chemical substance that is in liquid form at ambient temperature and atmospheric pressure and is both hydrophobic and lipophilic.

In some aspects, the composition is in the form of a microemulsion. A microemulsion has a droplet size in micrometers (10⁻⁶).

In some aspects, the composition is in the form of a nanoemulsion. A nanoemulsion has a droplet size in nanometers (10⁻⁹).

In some embodiments, at least about 60% of the at least one compound of formula (I) is present in a dispersed phase. In some aspects, at least about 70% of the personal care composition is in the dispersed phase. In some aspects, at least about 80% of the personal care composition is in the dispersed phase. In some aspects, at least about 90% of the personal care composition is in the dispersed phase. In some aspects, at least about 95% of the personal care composition is in the dispersed phase.

In some aspects, between about 60% and about 95% of the personal care composition is in the dispersed phase. In some aspects, the percentage of the personal care composition in the dispersed phase is between about 60% and about 95%, about 60% and about 90%, about 60% and about 80% about 60% and about 70%, about 70% and about 95%, about 70% and about 90%, about 70% and about 80%, about 80% and about 95%, about 80% and about 90%, or about 90% and about 95%.

In some aspects, the personal care composition is anhydrous. As used herein, the term “anhydrous” means that there is less than 2% by weight of water added to a composition based on the total weight of the composition. In some aspects, the compositions can comprise less than about 2% by weight of water, less than about 1% by weight of water, less than about 0.5% by weight of water, or less than 0.4% by weight of water.

In some aspects, the personal care composition is for application to a keratinous substrate, to a fabric, or to leather. In some aspects, the personal care composition is capable of forming a film on a keratinous substrate. In some aspects, the personal care composition is capable of forming a film on a keratinous substrate, wherein the film is capable of staying on the keratinous substrate without flaking or transferring to an object that comes in contact with the film.

In some aspects, the keratinous substrate is a feather, a fabric, a collagen, a hair, a nail, a skin, or a lip. In some aspects, the keratinous substrate is skin or lips.

In some aspects, the personal care composition shows a resistance to oil of at least 50%. In some aspects, the personal care composition shows a resistance to oil of at least 70%. In some aspects, the personal care composition shows a resistance to oil of at least 90%. In some aspects, the personal care composition shows a resistance to food oil of at least 50%. In some aspects, the personal care composition shows a resistance to food oil of at least 70%. In some aspects, the personal care composition shows a resistance to food oil of at least 90%. As used herein, the phrase “food oil” is an oil commonly used in cooking. In some aspects, the food oil is a vegetable oil, a soybean oil, a fish oil, a peanut oil, an avocado oil, a palm oil, an olive oil, a sesame oil, a corn oil, a coconut oil, or combinations thereof.

### III. Personal Care Formulations

In some aspects, the present disclosure is directed to a personal care formulation comprising at least one terpene polymer or the composition of at least one terpene polymer.

In some aspects, a personal care formulation is prepared by combining a personal care composition or a composition comprising at least one terpene polymer with at least one solvent to form a solid, gel, a paste, or a powder. In some aspects, the solvent is selected from the group consisting of water, an alcohol, a ketone, an ether, and an ester.

In some aspects, the personal care formulation is a solid. In some aspects, the personal care formulation is a gel. In some aspects, the personal care formulation is a paste. In some aspects, the personal care formulation is a powder.

In some aspects, a personal care formulation can be prepared by shearing the composition comprising at least one terpene polymer, as described herein, with a solvent, as described herein, to form a sheared gel. In some aspects, a personal care formulation can be prepared by combining a personal care composition or a composition comprising at least one terpene polymer, as described herein, with a solvent thereby forming a mixture and shearing the mixture.

In some aspects, the personal care formulation comprising at least one terpene polymer can be crumbled to form a powder.

In some aspects, the personal care formulation forms a film when applied to skin or lips.

In some aspects, the personal care formulation is selected from the group consisting of a deodorant, an antiperspirant, a skin cream, a facial cream, a hair shampoo, a hair conditioner, a mousse, a hair styling gel, a hair spray, a protective cream, a lipstick, a lip gloss, a lip color, a liquid lip, a facial foundation, a blush, a makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.

In some aspects, the personal care formulation is a beach sunscreen or a sports sunscreen.

In some aspects, the personal care formulation is a lipstick, a lip gloss, a lip color, or a liquid lip.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: Preparation of Poly(β-Pinene) with Molecular Weight ≤3,000 g/Mol

AlCl₃ (0.3 grams, 0.0022 mol) was mixed with 1.9 mL of dichloromethane and 0.4 mL of Ph₂O and stirred at room temperature for 20 minutes. A solution of 45 grams of 0-pinene in 45 grams of toluene was prepared and cooled to −30° C. in a 250 mL three-neck round bottle flask, which was equipped with an over-head stirrer, a thermocouple, and nitrogen gas. Then the AlCl₃ catalyst solution was quickly added to the three-neck round bottle flask with the β-pinene. The reaction mixture was then quenched with 1 M aqueous HCl. The organic layer was washed with water. The final product solution was dried under reduced pressure and a white powder was obtained. The resultant poly(β-pinene) had a weight average molecular weight of about 2,641 g/mol. The weight average molecular weight was determined by gel permeation chromatography with an evaporative light scattering detector (GPC-ELSD, Agilent Infinity 1260, Agilent Technologies, Inc., Waldbronn, Germany) in CH₂Cl₂ based on polystyrene narrow molecular weight standard calibration curves with a range of 1,000-100,000 Daltons.

### Example 2: Preparation of Poly(β-Pinene) with Molecular Weight ≥3,000 g/Mol

AlCl₃ (1.5 grams, 0.0112 mol) was mixed with 9.4 mL of dichloromethane and 1.8 mL of Ph₂O in a 250 mL three-neck round bottle flask, which was equipped with an over-head stirrer, a thermocouple, and nitrogen gas. The mixture was stirred at room temperature for 20 minutes. Then 225 g of toluene was added and the solution was cooled to 10° C. To the cooled toluene solution, 225 grams of β-pinene was added. The reaction mixture was then quenched with 1 M aqueous HCl. The resulting solution was transferred to a separation funnel and the organic layer was washed with water. The final product solution was dried under reduced pressure and a white powder was obtained. The resultant poly(β-pinene) had a weight average molecular weight greater than 3,000 g/mol with poly(β-pinene). Poly(β-pinene) with a weight average molecular weight of about 5,739 g/mol and poly(β-pinene) with a weight average molecular about 8,291 g/mol was obtained by this method. The weight average molecular weights were determined by GPC-ELSD (in CH₂Cl₂ based on polystyrene narrow molecular weight standard calibration curves with a range of 1,000-100,000 Daltons).

### Example 3: Preparation of Poly(Limonene)

Poly(limonene) was obtained using the synthetic method disclosed in Lyubushkin, A., et al., ChemEngineering 7(1):11 pages (2023).

### Formulation Example 1: Preparation of Cosmetic Formulations

Cosmetic formulations using terpene polymer compositions were prepared according to TABLE 5. Formulation 1 is a benchmark formulation that does not comprise a terpene polymer but does comprise a dimethicone gum and a silicone resin. Formulation 2 comprises poly(β-pinene) having a weight average molecular weight of about 8,291 g/mol that was prepared as described in Example 2 and also comprises a dimethicone gum and a silicone resin. Formulation 3 is a blank formulation that does not comprise a terpene polymer, does not comprise a dimethicone gum, and does not comprise a silicone resin. Formulation 4 comprises poly(β-pinene) having a weight average molecular weight of about 5,739 g/mol that was prepared as described in Example 2 and that also comprises a dimethicone gum and a silicone resin. Formulation 5 comprises poly(β-pinene) having a weight average molecular weight of about 2,641 g/mol that was prepared as described in Example 1 and that also comprises a dimethicone gum and a silicone resin. Formulation 6 comprises poly(limonene) that was prepared as described in Example 3 and also comprises a dimethicone gum and a silicone resin. Formulation 7 is a blank formulation that does not comprise a terpene polymer, does not comprise a dimethicone gum, and does not comprise a silicone resin.

TABLE 5
Cosmetic Formulations Prepared and Tested for Transfer Resistance

| | Formulation | | Formulation | | | | Formulation |
|---|---|---|---|---|---|---|---|
| | 1 | Formulation | 3 | Formulation | Formulation | Formulation | 7 |
| | (BM) | 2 | (Blank) | 4 | 5 | 6 | (Blank) |
| | (grams) | (grams) | (grams) | (grams) | (grams) | (grams) | (grams) |
| BENTONE GEL ® ISD V¹ | 19.05 | 9.525 | 19.05 | 19.05 | 19.05 | 9.525 | 9.525 |
| Red Lake 7² | 7.5 | 3.75 | 7.5 | 7.5 | 7.5 | 3.75 | 3.75 |
| TiO₂ MT-100TV³ | 1.47 | 0.735 | 1.47 | 1.47 | 1.47 | 0.735 | 0.735 |
| Isododecane | 9.47 | 8.18 | 21.98 | 16.98 | 16.98 | 8.49 | 10.99 |
| SE30⁴ | 5.63 | | | | | | |
| SR1000⁵ | 6.88 | | | | | | |
| Poly(β-pinene)⁵ with M_{W} = ~8,291 g/mol | | 2.81 | | | | | |
| Poly(β-pinene) with M_{W} = ~5,739 g/mol | | | | 5 | | | |
| Poly(β-pinene) with M_{W} = ~2,647 g/mol | | | | | 5 | | |
| Polylimonene⁷ | | | | | | 2.5 | |
| Total | 50 | 25 | 50 | 50 | 50 | 25 | 25 |

¹BENTONE GEL ISD V (Elementis Specialties, East Windsor, NJ);
²CI 15850 (Sun Chemical Corporation, Parsippany, NJ);
³Micro Titanium Dioxide (MT-100TV (Tayca Corporation, Osaka, Japan));
⁴Dimethicone gum (SILSOFT SE30 (Momentive Performance Materials, Inc., Niskayuna, New York));
⁵SR1000 Resin (Momentive Performance Materials, Inc., Niskayuna, New York);
⁶Poly(β-pinene) with M_{W} = ~8,291 g/mol was a 25.6% solution in isododecane; and
⁷Poly(limonene) from Example 3.

### Formulation Example 2: Transfer Resistance In Vitro Test of Cosmetic Formulations

The formulations prepared in Formulation Example 1 were subjected to the following transfer resistance in-vitro test protocol:

(a) Transfer resistance (TR): two types of substrates are used in this protocol: Vitro-Skin N19 (IMS Testing Group, Bunnell, FL) and Black Scrub Panel Paper P121-10N (BYK-Gardner GmbH, Geretsried, Germany). (1) Substrate 1 preparation: Vitro-Skin N19 was hydrated using 140 grams of distilled water and 60 grams of glycerin. Once hydrated overnight, it was placed in the center of a cutout of a previously prepared printing stencil, with the rough side to the top, then flattened against the bottom piece of sheeting to remove entrapped air and wrinkles. Then a sample from the tested formulation was placed in an approximately 0.5 mm thick line along the left side of the VITRO-SKIN. Using the 1 mm gap side of the wet film applicator, a uniform coating of sample over the entire surface of the VITRO-SKIN was achieved by slowly pulling the wet film applicator over the flat VITRO-SKIN. Then, the so-coated VITRO-SKIN was placed in a 50° C. oven for 30 minutes. (2) Substrate 2 preparation: Black Scrub Panel Paper P121-10N) was coated with a 3 mil film using a wet film applicator, and then subjected to 50° C. drying for 10 min in an oven. Then the substrate (1) or substrate (2) was placed on a clean flat surface. Using a rubber band, a piece of transfer cloth was tightly attached to the bottom of the 500 gm weight. The weight was centered on the coated piece of substrate (1) or substrate (2), which was securely held in place while the weight was rotated in small steps through 360° by moving it through as many partial rotations as needed to make an entire circle. Then the cotton cloth was removed from the weight and visually examined for the degree of color transfer.

(b) Water resistance (WR): The same process as in (a) was employed, except that prior to the cloth-covered weight application, 9 drops of distilled water were applied onto the center of the coated and dried substrate, and after 1 minute the cotton cloth was applied as described above.

(c) Food oil resistance (OR): Same procedure as (a) and (b) but instead of water, corn oil was used.

The results are depicted in FIGS. 1-5, where a lighter color indicates less color transfer from the substrate to the cotton cloth. As shown in the FIGs, Formulation 2 (comprising poly(β-pinene) with a M_{W}=˜8,291 g/mol) and Formulation 4 (comprising poly(β-pinene) with a M_{W}=˜5,739 g/mol) showed good transfer resistance, water resistance, and food oil resistance at the same time, compared to the blank (Formulation 3). In particular, the transfer resistance, water resistance, and food oil resistance tests for Formulation 2 showed very little transference. The transfer resistance and water resistance tests for Formulation 5 (comprising poly(β-pinene) with a M_{W}=˜2,641 g/mol) showed very little transference; however, the increased transfer to a cotton cloth for the food oil resistance test indicates that having a molecular weight >2,641 g/mol is important for performance.

Formulation 6 (comprising poly(limonene)) did not show an improvement in food oil resistance compared to the blank (Formulation 7), which did not comprise a film former.

Food oil resistance is reflected by color transfer. Those values, measured in the CIELAB color space (averages of three readings with an X-Rite COLOR-EYE 7000A spectrophotometer (X-Rite, Inc., Grand Rapids, MI) white cotton gives a value of 1.58)), are shown in TABLE 6. The lower the number, the less color has transferred, hence the better the transfer resistance. Transfer resistance and food oil resistance values were also measured for Formulations 2 and 4-6 and provided values ranging from 6.15 to 27.84.

TABLE 6
Food Oil Resistance Values for Tested Formulations

| | | | Food Oil |
|---|---|---|---|
| Film Former | Formulation | Color Transfer | Resistance [%] |
| Poly(β-pinene) with | Formulation 2 | 8.34 | 73 |
| Mw = ~8,291 g/mol | | | |
| Poly(β-pinene) with | Formulation 4 | 6.15 | 80 |
| Mw = ~5,739 g/mol | | | |
| Poly(β-pinene) with | Formulation 5 | 25.14 | 19 |
| Mw = ~2,641 g/mol | | | |
| Poly(limonene) | Formulation 6 | 27.84 | 10 |

### Formulation Example 3: Presence of Plasticizers

TABLE 7 shows formulations where various plasticizing oils or gums were added to the film former containing formulations.

TABLE 7
Formulations with Plasticizing Oils or Gums

| | Formulation 8 | Formulation 9 | Formulation 10 | Formulation 11 | Formulation 12 | Formulation 13 |
|---|---|---|---|---|---|---|
| | (grams) | (grams) | (grams) | (grams) | (grams) | (grams) |
| Bentone Gel | 9.525 | 9.525 | 9.525 | 9.525 | 9.525 | 9.525 |
| ISDV¹ | | | | | | |
| Red Lake 7² | 3.75 | 3.75 | 3.75 | 3.75 | 3.75 | 3.75 |
| TiO₂ MT- | 0.735 | 0.735 | 0.735 | 0.735 | 0.735 | 0.735 |
| 100TV³ | | | | | | |
| Permethyl ® 99A⁴ | 7.99 | 7.865 | 7.865 | 7.865 | 7.865 | 7.865 |
| Poly(β-pinene) | 2.5 | | | | | |
| Mw~11516 | | | | | | |
| g/mol | | | | | | |
| Poly(β-pinene) | | 2.5 | 1.875 | 2.5 | 1.875 | 2.5 |
| Mw~7091 | | | | | | |
| g/mol | | | | | | |
| PIB 1499⁵ | 0.5 | | | | | |
| LEXOREZ 100 | | 0.625 | 1.25 | | | |
| MB⁶ | | | | | | |
| Triethyl Citrate⁷ | | | | 0.625 | 1.25 | |
| HARMONIE | | | | | | 0.625 |
| NatuDerma⁸ | | | | | | |
| Polyester | | | | | | |
| elastomer gel⁹ | | | | | | |
| Poly(β-pinene) of | | | | | | |
| Example 2 | | | | | | |
| SR1000¹⁰ | | | | | | |
| SE30¹¹ | | | | | | |
| Total | 25 | 25 | 25 | 25 | 25 | 25 |

| | | | | | no Film | |
|---|---|---|---|---|---|---|
| | Formulation 14 | Formulation 15 | Formulation 16 | Formulation 17 | Former | |
| | (grams) | (grams) | (grams) | (grams) | (grams) | MQ/GUM |
| Bentone Gel | 9.525 | 9.525 | 9.525 | 9.525 | 9.525 | 9.525 |
| ISDV¹ | | | | | | |
| Red Lake 7² | 3.75 | 3.75 | 3.75 | 3.75 | 3.75 | 3.75 |
| TiO₂ MT- | 0.735 | 0.735 | 0.735 | 0.735 | 0.735 | 0.735 |
| 100TV³ | | | | | | |
| Permethyl ® 99A⁴ | 7.865 | 7.865 | 7.865 | 7.865 | 10.99 | 4.735 |
| Poly(β-pinene) | | | | | | |
| Mw~11516 | | | | | | |
| g/mol | | | | | | |
| Poly(β-pinene) | 2.5 | 1.875 | 2.5 | 1.875 | | |
| Mw~7091 | | | | | | |
| g/mol | | | | | | |
| PIB 1499⁵ | | | | | | |
| LEXOREZ 100 | | | | | | |
| MB⁶ | | | | | | |
| Triethyl Citrate⁷ | | | | | | |
| HARMONIE | | | | | | |
| NatuDerma⁸ | | | | | | |
| Polyester | 0.625 | 1.25 | | | | |
| elastomer gel⁹ | | | | | | |
| Poly(β-pinene) of | | | 0.625 | 1.25 | | |
| Example 2 | | | | | | |
| SR1000¹⁰ | | | | | | 3.44 |
| SE30¹¹ | | | | | | 2.815 |
| Total | 25 | 25 | 25 | 25 | 25 | 25 |

¹BENTONE GEL ISD V (Elementis Specialties, East Windsor, NJ);
²CI 15850 (Sun Chemical Corporation, Parsippany, NJ);
³Micro Titanium Dioxide (MT-100TV (Tayca Corporation, Osaka, Japan));
⁴Isododecane (Presperse Corp., Somerset, NJ);
⁵Polybutenes (Sigma-Aldrich, Inc., St. Louis, MO);
⁶Adipic Acid/Diglycol Crosspolymer (Inolex (Philadephia, PA));
⁷(Vigon-Azelis (East Stroudsburg, PA));
⁸C48-Isoalkane, Polyester Elastomer (Momentive Performance Materials, Inc., Niskayuna, New York);
⁹Dodecane, Epoxydized Soybean Oil (Momentive Performance Materials, Inc., Niskayuna, New York);
¹⁰SR1000 Resin (Momentive Performance Materials, Inc., Niskayuna, New York);
¹¹Dimethicone gum (SILSOFT SE30 (Momentive Performance Materials, Inc., Niskayuna, New York)).

The formulations in TABLE 7 comprising plasticizing oils and gums showed good transfer-, water- and food-oil-resistance performance, especially when compared to the blank (no film former), and also when compared to the benchmark (MQ/gum).

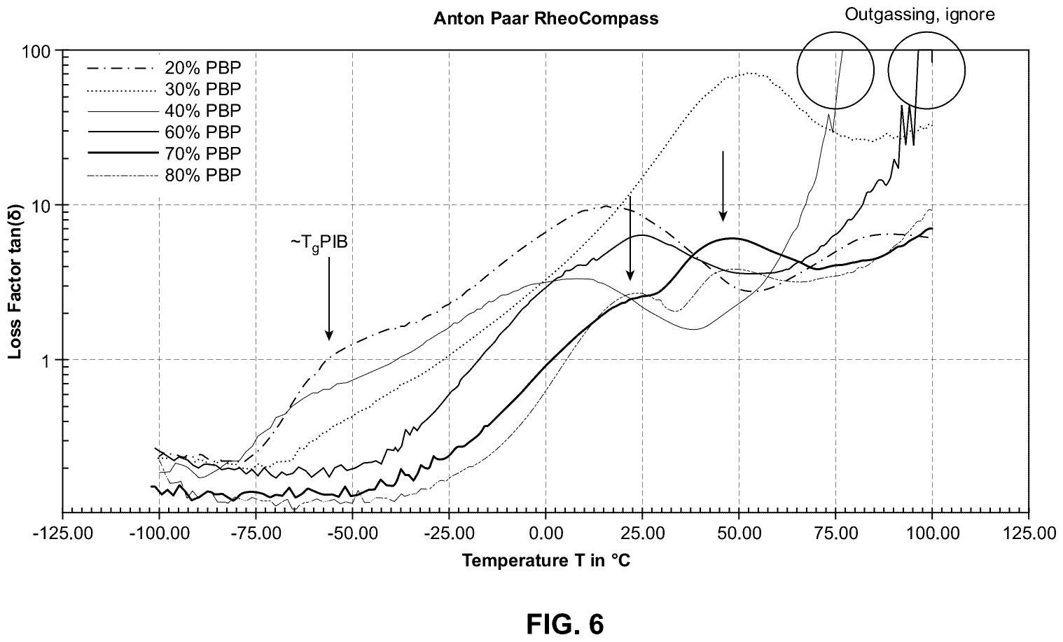

In order to confirm plasticization actually occurred, dynamic mechanical analysis (DMA) experiments were conducted using different plasticizer/film former (poly(β-Pinene) ((PBP)) ratios. FIG. 6 shows the DMA curves obtained as a function of temperature using polybutene (also known as polyisobutylene (PIB) as the plasticizer.

As can be seen from, for example, the two peaks in FIG. 6 at 70% (green curve) and 80% (orange curve), PBP concentration, at ˜20° C. and ˜45° C., respectively, confirms that new glass transitions (Tg's) are occurring, which is strong evidence for a plasticization effect, as opposed to, for example, a mere solubilization effect.

### Formulation Example 4: Pigments & Dyes

TABLE 8 shows the components in Formulation 18 which include untreated pigments. For Formulation 18, the presence of poly(ß-pinene) provided exceptionally low color transfer performance.

TABLE 8
Formulation of Poly(β-pinene) with Untreated Pigments

| | Formulation |
|---|---|
| | 18 (grams) |
| Bentone Gel ISD | 9.525 |
| Suncroma Red Iron Oxide | 2.75 |
| Suncroma Black Iron Oxide | 0.1 |
| Suncroma Yellow Iron Oxide | 0.9 |
| TiO2 MT100TV | 0.735 |
| IDD | 8.49 |
| POLY(β-PINENE) EXAMPLE 2 | 2.5 |
| Total | 25 |

TABLE 9 shows the ingredients in Formulations 19-21 which include treated pigment systems and two different dyes. For Formulations 19-21, the presence of poly(8-pinene) provided the desired low color transfer performance.

TABLE 9
Formulations with Poly(β-pinene) and Treated Pigments

| | Formulation | Formulation | Formulation |
|---|---|---|---|
| | 19 (grams) | 20 (grams) | 21 (grams) |
| Bentone Gel ISD | 9.525 | 9.525 | 9.525 |
| Kobo BWRO-ASGP3 (red) | 2.75 | 1.375 | 1.375 |
| Kobo BWBO-ASGP3 (black) | 0.1 | 0.05 | 0.05 |
| Kobo BYO-ASGP3 (yellow) | 0.9 | 0.45 | 0.45 |
| Red No 28 AL | | 1.875 | |
| Lake (CI 45410) | | | |
| DC Red Lake 7 (CI 15850) | | | 1.875 |
| Kobo BTD-ASGP3 (white) | 0.735 | 0.735 | 0.735 |
| IDD | 8.49 | 8.49 | 8.49 |
| POLY(β-PINENE) | 2.5 | 2.5 | 2.5 |
| EXAMPLE 2 | | | |
| Total | 25 | 25 | 25 |

### Formulation Example 5: Thickeners and Rheology Modifiers

TABLE 10 shows formulations with different thickeners/rheology modifiers used.

TABLE 10
Formulations of Poly(ß-pinene) with Thickeners/Rhelology Modifiers

| | Formulation | Formulation | Formulation | Formulation | Formulation | Formulation |
|---|---|---|---|---|---|---|
| | 22 | 23 | 24 | 25 | 26 | 27 |
| | (grams) | (grams) | (grams) | (grams) | (grams) | (grams) |
| Bentone Gel ISD | 5.525 | 5.525 | 6.525 | 6.525 | 6.525 | 6.525 |
| DC Red Lake 7 (CI | 3.75 | 3.75 | | | | |
| 15850) | | | | | | |
| Red Iron Oxides CI | | | 3.75 | | | |
| 77491 | | | | | | |
| Yellow Iron | | | | 3.75 | | |
| Oxides CI 77492 | | | | | | |
| Black Iron Oxides | | | | | 3.75 | 3.75 |
| CI 77499 | | | | | | |
| TiO2 MT100TV | 0.735 | 0.735 | 0.735 | 0.735 | 0.735 | 0.735 |
| IDD | 8.49 | 8.49 | 8.49 | 8.49 | 8.49 | 8.49 |
| Fumed Silica | 4 | | | | | |
| Calcium Sodium | | 4 | | | | |
| Borosilicate | | | | | | |
| Kaolin | | | 3 | 3 | 3 | 3 |
| POLY(ß- | 2.5 | 2.5 | 2.5 | 2.5 | 2.5 | 2.5 |
| PINENE) | | | | | | |
| EXAMPLE 2 | | | | | | |
| Total | 25 | 25 | 25 | 25 | 25 | 25 |

### Further Formulation Examples

The following formulation examples are illustrative, but not limiting, of the methods described herein. Other suitable modifications and adaptations of the variety of conditions and parameters normally encountered in the field, and which are obvious to those skilled in the art, are within the spirit and scope of the invention.

### 1. Color Cosmetics Formulation Examples

TABLE 11 provides formulation details for a natural longwear liquid lip.

TABLE 11
Natural Longwear Liquid Lip Formulation

| Phase | Raw Material | INCI Name | % W/W |
|---|---|---|---|
| A | Permethyl ® 99A | Isododecane | 20.00 |
| | POLY(β-PINENE) EXAMPLE 2 | Polypinene | 10.00 |
| B | SustOleo DCS | Diisooctyl Succinate | 8.00 |
| | SunCROMA ® D&C Red 7 Ca | Red 7 Lake | 8.00 |
| | Lake | | |
| | BTD-ASGP3 | Titanium Dioxide (And) Stearoyl | 4.00 |
| | | Glutamic Acid (And) | |
| | | Polyhydroxystearic Acid | |
| | BWBO-ASGP3 | Iron Oxides (CI 77499) (And) | 0.50 |
| | | Stearoyl Glutamic Acid (And) | |
| | | Polyhydroxystearic Acid | |
| | BWYO-ASGP3 | Iron Oxides (CI 77492) (And) | 1.50 |
| | | Stearoyl Glutamic Acid (And) | |
| | | Polyhydroxystearic Acid | |
| C | ImerCare 06K | Kaolin | 13.00 |
| | Sericite GMS-4C | Mica | 10.00 |
| | Harmonie Luxe 4 Powder | Silica | 5.00 |
| | BENTONE GEL ® ISD V | Isododecane (and) Disteardimonium | 20.00 |
| | | Hectorite (and) Propylene Carbonate | |

TABLE 12 provides formulation details for a natural longwear lipstick.

TABLE 12
Formulation for a Natural Longwear Lipstick

| Phase | Ingredients | INCI Name | Wt (%) |
|---|---|---|---|
| A | MCT Oil | Capric/Caprylic Triglyceride | 20.00 |
| | BTD-ASGP3 | Titanium Dioxide (And) Stearoyl | 9.85 |
| | | Glutamic Acid (And) | |
| | | Polyhydroxystearic Acid | |
| | BWRO-ASGP3 | Iron Oxides (CI 77491) (And) | 4.00 |
| | | Stearoyl Glutamic Acid (And) | |
| | | Polyhydroxystearic Acid | |
| | BWYO-ASGP3 | Iron Oxides (CI 77492) (And) | 0.40 |
| | | Stearoyl Glutamic Acid (And) | |
| | | Polyhydroxystearic Acid | |
| | BWBO-ASGP3 | Iron Oxides (CI 77499) (and) | 0.75 |
| | | Stearoyl Glutamic Acid (and) | |
| | | Polyhydroxystearic Acid | |
| B | Candelilla Wax NP | Euphorbia Cerifera (Candelilla) | 5.00 |
| | | Wax | |
| | Carnauba Wax | Copernicia Cerifera (Carnauba) | 5.00 |
| | | Wax | |
| | Kester Wax K 60P | Polyhydroxystearin Wax | 5.00 |
| C | Harmonie Natu Vel Gel | Coco-Caprylate/Caprate (and) | 10.00 |
| | | Polyglyceryl-2 Isostearate/Dimer | |
| | | Dilinoleate Copolymer (and) | |
| | | Squalane | |
| | Harmonie Luxe 4 Powder | Silica | 10.00 |
| D | Permethyl ® 99A | Isododecane | 10.00 |
| | POLY(β-PINENE) EXAMPLE 2 | Polypinene | 20.00 |

TABLE 13 provides formulation details for a natural-ish eyeliner.

TABLE 13
Formulation for a Natural-ish Eyeliner

| Phase | Raw Material | INCI Name | % W/W |
|---|---|---|---|
| A | Permethyl ® 99A | Isododecane | 30.00 |
| | POLY(β-PINENE) EXAMPLE 2 | Polypinene | 10.00 |
| B | Rice Bran Wax | Rice Bran Wax | 6.00 |
| C | Kester Wax K-60P | Polyhydroxystearic Acid | 4.00 |
| | Bentone ISD V | Isododecane (and) Disteardimonium | 20.00 |
| | | Hectorite (and) Propylene Carbonate | |
| D | BWBO-ASPG3 | Iron Oxides (CI 77499) (And) | 20.00 |
| | | Stearoyl Glutamic Acid (And) | |
| | | Polyhydroxystearic Acid | |
| | MCT Oil | Caprylic/Capric Triglyceride | 10.00 |

TABLE 14 provides formulation details for a sparkling eyeshadow.

TABLE 14
Formulation for a Sparkling Eyeshadow

| Phase | Raw Material | INCI Name | % W/W |
|---|---|---|---|
| A | Permethyl ® 99A | Isododecane | 33.00 |
| | POLY(β-PINENE) EXAMPLE 2 | Polypinene | 10.00 |
| | Kester Wax K-60P | Polyhydroxystearic Acid | 2.00 |
| | Syncrowax ™ HRC | Tribehenin | 2.00 |
| | Bentone ISD V | Isododecane (and) Disteardimonium | 13.00 |
| | | Hectorite (and) Propylene Carbonate | |
| D | Sericite GMS-4C | Mica | 10.00 |
| | K-Ray SM Nude | Synthetic Fluorphlogopite (And) | 15.00 |
| | | Titanium Dioxide (And) Iron Oxides | |
| | | (CI 77491) | |
| | K-Ray MagBrown | Mica (And) Iron Oxides (CI 77491) | 5.00 |
| | | (And) Iron Oxides (CI 77499) | |
| | Harmonie Luxe 4 Powder | Silica | 10.00 |

TABLE 15 provides formulation details for a natural foundation.

TABLE 15
Formulation for a Natural Foundation

| Phase | Raw Material | INCI Name | % W/W |
|---|---|---|---|
| A | DI Water | Water | 42.00 |
| | Glycerin | Glycerin | 1.00 |
| | Sodium Chloride | Sodium Chloride | 1.00 |
| | Magnesium Sulfate | Magnesium Sulfate | 1.00 |
| B | Harmonie Soft Fluid | C9-12 Alkane | 20.00 |
| | POLY(β-PINENE) EXAMPLE 2 | Polypinene | 10.00 |
| | Emulium ® Illustro | Polyglyceryl-6 Polyhydroxystearate | 5.00 |
| | | (and) Polyglyceryl-6 Polyricinoleate | |
| C | MCT Oil | Caprylic/Capric Triglyceride | 10.00 |
| | BTD-ASGP3 | Titanium Dioxide (And) Stearoyl | 8.30 |
| | | Glutamic Acid (And) | |
| | | Polyhydroxystearic Acid | |
| | BWRO-ASGP3 | Iron Oxides (CI 77491) (And) | 0.50 |
| | | Stearoyl Glutamic Acid (And) | |
| | | Polyhydroxystearic Acid | |
| | BWYO-ASGP3 | Iron Oxides (CI 77492) (And) | 1.04 |
| | | Stearoyl Glutamic Acid (And) | |
| | | Polyhydroxystearic Acid | |
| | BWBO-ASGP3 | Iron Oxides (CI 77499) (And) | 0.16 |
| | | Stearoyl Glutamic Acid (And) | |
| | | Polyhydroxystearic Acid | |

TABLE 16 provides formulation details for a long-lasting mascara.

TABLE 16
Formulation for a Long-Lasting Mascara

| Phase | Raw Material | INCI Name | % W/W |
|---|---|---|---|
| A | Water | Aqua | 52.50 |
| | Xanthan Gum | Xanthan Gum | 1.00 |
| B | Harmonie Soft fluid | C9-12 Alkane | 10.00 |
| | POLY(β-PINENE) EXAMPLE 2 | Polypinene | 10.00 |
| | Synchrowax HRC-PA | Tribehenin | 10.00 |
| | Lexemul 515 MB | Glyceryl Stearate | 2.50 |
| | Lanette O NA | Cetearyl Alcohol | 3.00 |
| C | BGBO - TTB2 | Iron Oxide (CI 77499) (and) | 10.00 |
| | | Isopropyl Titanium Triisostearate | |
| | | (and) Triethoxysilylethyl | |
| | | Polydimethylsiloxyethyl | |
| | | Dimethicone | |
| C | S&M Phenoxyethanol RCH | Phenoxyethanol | 1.00 |

TABLE 17 provides formulation details for a long-lasting eyeliner.

TABLE 17
Formulation for a Long- lasting Eyeliner

| Phase | Raw Material | INCI Name | % W/W |
|---|---|---|---|
| A | Bentone Gel ISD V | Isododecane (and) | 38.10 |
| | | Disteardimonium Hectorite (and) | |
| | | Propylene Carbonate | |
| | Permethyl ® 99A | Isododecane | 25.64 |
| B | POLY(β-PINENE) EXAMPLE 2 | Polypinene | 5.00 |
| C | BGBO - TTB2 | Iron Oxide (CI 77499) (and) | 15.00 |
| | | Isopropyl Titanium Triisostearate | |
| | | (and) Triethoxysilylethyl | |
| | | Polydimethylsiloxyethyl | |
| | | Dimethicone | |
| | K - RAY SM FINE BLACK | Iron Oxides (CI 77499) (And) | 5.00 |
| | | Synthetic Fluorphlogopite (And) | |
| | | Titanium Dioxide | |
| D | SilSoft SE 30 Gum | Dimethicone | 10.26 |
| | S&M Phenoxyethanol RCH | Phenoxyethanol | 1.00 |

### 2. Examples of Sun Formulations

TABLE 18 provides formulation details for an anhydrous sun spray.

TABLE 18
Formulation for an Anhydrous Sun Spray*

| Phase | Raw Material | INCI Name | % W/W |
|---|---|---|---|
| A | Avobenzone | Avobenzone | 3.00 |
| | Parsol EHS | Ethylhexyl Salicylate | 5.00 |
| | Parsol 340 | Octocrylene | 10.00 |
| | Parsol HMS | Homosalate | 10.00 |
| | C12-15 Alkyl Benzoate | C12-15 Alkyl Benzoate | 10.00 |
| B | POLY(β-PINENE) | Polypinene | 10.00 |
| | EXAMPLE 2 | | |
| | Cetiol ® C 5C | Coco-Caprylate/Caprate | 25.00 |
| | Harmonie Soft Fluid | C9-12 Alkane | 27.00 |

TABLE 19 provides formulation details for a sun cream.

TABLE 19
Formulation for a Sun Cream*

| Phase | Raw Material | INCI Name | % W/W |
|---|---|---|---|
| A | Avobenzone | Avobenzone | 3.00 |
| | Parsol EHS | Ethylhexyl Salicylate | 5.00 |
| | Parsol 340 | Octocrylene | 10.00 |
| | Parsol HMS | Homosalate | 10.00 |
| B | POLY(β-PINENE) EXAMPLE 2 | Polypinene | 10.00 |
| | Cetiol ® C 5C | Coco-Caprylate/Caprate | 10.00 |
| | Harmonie Soft Fluid | C9-12 Alkane | 10.00 |
| | BENTONE ® LUXE XO | C13-15 Alkane (and) | 10.00 |
| | | Disteardimonium Hectorite (and) | |
| | | Polyglyceryl-3 Polyricinoleate | |
| C | DI Water | DI Water | 28.80 |
| | Glycerin | Glycerin | 1.00 |
| | Spectrastat G2 Natural MB | Caprylhydroxamic Acid (and) | 1.20 |
| | | Glyceryl Caprylate (and) Glycerin | |
| | Sodium Chloride | Sodium Chloride | 1.00 |

TABLE 20 provides formulation details for an SPF makeup finishing spray.

TABLE 20
Formulation for a SPF Makeup Finishing Spray

| Phase | Raw Material | INCI Name | (%)W/W |
|---|---|---|---|
| A | Avobenzone | Avobenzone | 3.00 |
| | Parsol EHS | Ethylhexyl Salicylate | 5.00 |
| | Parsol 340 | Octocrylene | 10.00 |
| | Parsol HMS | Homosalate | 10.00 |
| | C12-15 Alkyl Benzoate | C12-15 Alkyl Benzoate | 10.00 |
| B | POLY(β-PINENE) EXAMPLE 2 | Polypinene | 5.00 |
| | Harmonie Luxe 4 Powder | Silica | 5.00 |
| | Cetiol ® C 5C | Coco-Caprylate/Caprate | 20.00 |
| | Permethyl ® 99A | Isododecane | 32.00 |

### 3. Examples of Skin Formulations

TABLE 21 provides formulation details for a lightweight cream.

TABLE 21
Formulation for a Lightweight Cream

| Phase | Ingredients | INCI Name | Wt (%) |
|---|---|---|---|
| A | DI Water | DI Water | 46.80 |
| | Glycerin | Glycerin | 1.00 |
| | Spectrastat G2 Natural MB | Caprylhydroxamic Acid (and) | 1.20 |
| | | Glyceryl Caprylate (and) Glycerin | |
| | Sodium Chloride | Sodium Chloride | 1.00 |
| B | Permethyl ® 99A | Isododecane | 20.00 |
| | POLY(β-PINENE) EXAMPLE 2 | Polypinene | 10.00 |
| | BENTONE ® LUXE XO | C13-15 Alkane (and) | 10.00 |
| | | Disteardimonium Hectorite (and) | |
| | | Polyglyceryl-3 Polyricinoleate | |
| | Cetiol ® C 5C | Coco-Caprylate/Caprate | 10.00 |

### OTHER ASPECTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed.

1. A personal care composition comprising at least one terpene polymer with a weight average molecular weight greater than 3,000 g/mol.

2. The personal care composition of claim 1, wherein the at least one terpene polymer is obtained by polymerizing a terpene monomer selected from the group consisting of a monoterpene, a sesquiterpene, a diterpene, a sesterterpene, a triterpene, a sesquaterpene, a tetraterpene, and combinations thereof.

3. The personal care composition of claim 2, wherein the terpene monomer is a monoterpene selected from the group consisting of myrcene, ocimene, alloocimene, L-limonene, D-limonene, α-pinene, and β-pinene.

4. The personal care composition of claim 1, wherein the at least one terpene polymer is a homopolymer obtained by polymerizing a terpene monomer selected from the group consisting of α-pinene, β-pinene, L-limonene, and D-limonene.

5. The personal care composition of claim 1, wherein the at least one terpene polymer is a copolymer obtained by polymerizing at least one terpene monomer selected from the group consisting of α-pinene, β-pinene, L-limonene, D-limonene, and combinations thereof.

6. The personal care composition of claim 5, wherein at least one terpene monomer comprises at least 50% of the total weight of monomers used to obtain the copolymer.

7. (canceled)

8. The personal care composition of claim 1, wherein the at least one terpene polymer is a homopolymer of β-pinene.

9. (canceled)

10. The personal care composition of claim 1, wherein the at least one terpene polymer is a copolymer obtained by polymerizing at least one terpene monomer and at least one monomer that is not a terpene.

11. The personal care composition of claim 1, wherein the at least one terpene polymer has a weight average molecular weight from about 3,500 g/mol to about 100,000 g/mol.

12.-13. (canceled)

14. The personal care composition of claim 1, wherein the at least one terpene polymer has a glass transition temperature from about 50° C. to about 300° C.

15. (canceled)

16. The personal care composition of claim 1, wherein the personal care composition further comprises at least one volatile oil.

17.-18. (canceled)

19. The personal care composition of claim 1, wherein the personal care composition further comprises a pigment or a dye.

20. (canceled)

21. The personal care composition of claim 1, wherein the personal care composition further comprises a non-volatile oil.

22. (canceled)

23. The personal care composition of claim 1, wherein the personal care composition further comprises at least one elastomer.

24. The personal care composition of claim 23, wherein the elastomer is a crosslinked elastomer.

25.-26. (canceled)

27. The personal care composition of claim 1, wherein the personal care composition further comprises a wax.

28. The personal care composition of claim 1, wherein the personal care composition further comprises a rheology modifier.

29.-31. (canceled)

32. The personal care composition of claim 1, wherein the weight of the at least one terpene polymer in the composition is from about 0.1% to about 30% of the total weight of the composition.

33. The personal care composition claim 1, wherein the composition is capable of forming a film on a keratinous substrate.

34. The personal care composition of claim 33, wherein the film is capable of remaining on the keratinous substrate for at least one hour without flaking or transferring to an object that comes in contact with the film.

35. The personal care composition of claim 33, wherein the film has a resistance to food oil of greater than or equal to about 50% as measured by color transfer.

36. A personal care formulation comprising the personal care composition of claim 1, wherein the personal care formulation is a deodorant, an antiperspirant, a skin cream, a facial cream, a hair shampoo, a hair conditioner, a mousse, a hair styling gel, a hair spray, a protective cream, a lipstick, a lip color, a lip stain, a lip gloss, a facial foundation, a concealer, a blush, a makeup, a mascara, a BB cream, a CC cream, a skin care lotion, a moisturizer, a facial treatment, a personal cleanser, a facial cleanser, a bath oil, a perfume, a shaving cream, a pre-shave lotion, an after-shave lotion, a cologne, a sachet, or a sunscreen.

37.-38. (canceled)
